SEQUENCE LISTING (196 sequences)

SEQ ID NO: 1 - DNA, 1945 bp, Caenorhabditis elegans
  exon (1)...(83): Exon 9 of the Caenorhabditis elegains mec-2 gene.
agagctgaaaagaactcaacgattatattcccgttcccaattgatcttctcagtgcattcctccaacgaacaccgcctaaagtgtaagttttcacagagtattcgacaaaaagcacaatctattcctatcaaattgcagtgataacaattttgcatttccaacgcacaaagctggcggaataccgtcttcctcttgaacacttcacgaattcaaaattcattgacatgcgtgtgatcagccaatttcatttttccacatcgctttgagtgacctcacacccactgataataattgtcttactgcttcatttccatttttctcaaattccacataggagcgttagagttccattcaactggtaacagtcacacaaaaacacaaacttccctctgaacaaaaacatagtcataatcgtttgctgagtaatctcggtgtatcgtcaaattcaaccaacccatcctgtaaatcctcctgtcctgtcttttcaatagctctttttgacagtaacatttcatgttttgaaaaatgtgataaaccttcgatcccccaaacacttcttttcaattcttttgaacaatgttcaatacaaatttaacattgaatcttataagctttttttcacaaaaaacttgagttatatatagattatcaactttcttatttctttcaaataatcccttatccattatttttcaatgaattttatcatatcattgcttactgatttggcattttcttcttgaaattcgacaccaacactgggtaatacatgttgttctcgcacaaagagtatcgtatgttgcttcgactcgccacactgcttttctcaaatggatgaaacattttcgaaaattggaaaacctaaaccgcatacgagtgagtgcaaaaataaatttggcaaccacactacagcttcgattatcacttcacaaaaaataaaaacagggtcgacgaacttttttgaactggccaaaaacgtattctaaaaatgtcaaactctttaaattggaatatcacaaattacgcatactgaaatttaatgaagagctatgccgataaaatggaactacaacttatttgacgcttgtgtattaaagacacaatatttcaaaaaaattacgttcacatttagataaggggtatcctcagtagaaaacgtgttcaaaacctctactatagagtaatgttggaaattattaaatatttacaatttttcaaaatactctgtaatttaccaaacctttcactgaaaattttatcataatgttaattgtcgaccaacaatttgtttccaatgatttatatgtttacatttttagcgttttgataattcctatttgaacattacaattgttcaaaactacatcaatgctcgaaattccttagaatttcgcattttcagaaatttggcaatttaccacaactgtgattgattttttcaaaaacatttcgaaaaatcaaaaatgttttgggttggatagtttaaaaatttggtcttgttagactataaaaatatttagactatattcccactgacaatgcttcagattaaatcttgttattaatttcttctttttacgcttcttaagcacaaaaatacgaaaaaaaatcgtcaattaaaaaaaaattagaaatgatttctaataatatactaagagcgttttttaatcttggtatgtgttggaaatgagattcaatcaaatcttcaaaaaaatcacagggaacatttttgaagtttgtttcatttcactgaaacactattttttcattgaatagcaagttttcagtgaggagccaccgtctttaccgaaaaaaatccgttcatgctgcttgtacaagtaccctgattgggtgcaaggaatggttggatctgaaggtggtggaggtcacggacattcccatggaggaggaggaggagggcttggatcgtcacaggtgagggct

SEQ ID NO: 2 - DNA, 1705 bp, Caenorhabditis elegans
gtaagttttcacagagtattcgacaaaaagcacaatctattcctatcaaattgcagtgataacaattttgcatttccaacgcacaaagctggcggaataccgtcttcctcttgaacacttcacgaattcaaaattcattgacatgcgtgtgatcagccaatttcatttttccacatcgctttgagtgacctcacacccactgataataattgtcttactgcttcatttccatttttctcaaattccacataggagcgttagagttccattcaactggtaacagtcacacaaaaacacaaacttccctctgaacaaaaacatagtcataatcgtttgctgagtaatctcggtgtatcgtcaaattcaaccaacccatcctgtaaatcctcctgtcctgtcttttcaatagctctttttgacagtaacatttcatgttttgaaaaatgtgataaaccttcgatcccccaaacacttcttttcaattcttttgaacaatgttcaatacaaatttaacattgaatcttataagctttttttcacaaaaaacttgagttatatatagattatcaactttcttatttctttcaaataatcccttatccattatttttcaatgaattttatcatatcattgcttactgatttggcattttcttcttgaaattcgacaccaacactgggtaatacatgttgttctcgcacaaagagtatcgtatgttgcttcgactcgccacactgcttttctcaaatggatgaaacattttcgaaaattggaaaacctaaaccgcatacgagtgagtgcaaaaataaatttggcaaccacactacagcttcgattatcacttcacaaaaaataaaaacagggtcgacgaacttttttgaactggccaaaaacgtattctaaaaatgtcaaactctttaaattggaatatcacaaattacgcatactgaaatttaatgaagagctatgccgataaaatggaactacaacttatttgacgcttgtgtattaaagacacaatatttcaaaaaaattacgttcacatttagataaggggtatcctcagtagaaaacgtgttcaaaacctctactatagagtaatgttggaaattattaaatatttacaatttttcaaaatactctgtaatttaccaaacctttcactgaaaattttatcataatgttaattgtcgaccaacaatttgtttccaatgatttatatgtttacatttttagcgttttgataattcctatttgaacattacaattgttcaaaactacatcaatgctcgaaattccttagaatttcgcattttcagaaatttggcaatttaccacaactgtgattgattttttcaaaaacatttcgaaaaatcaaaaatgttttgggttggatagtttaaaaatttggtcttgttagactataaaaatatttagactatattcccactgacaatgcttcagattaaatcttgttattaatttcttctttttacgcttcttaagcacaaaaatacgaaaaaaaatcgtcaattaaaaaaaaattagaaatgatttctaataatatactaagagcgttttttaatcttggtatgtgttggaaatgagattcaatcaaatcttcaaaaaaatcacagggaacatttttgaagtttgtttcatttcactgaaacactattttttcattgaatagcaagttttcag

SEQ ID NO: 3 - protein, 37 aa, Artificial Sequence
  Synthetic polypeptide
VSFHRVFDKKHNLFLSNCSDNNFAFPTHKAGGIPSSS

SEQ ID NO: 4 - protein, 8 aa, Artificial Sequence
  Synthetic polypeptide
TLHEFKIH

SEQ ID NO: 5 - protein, 36 aa, Artificial Sequence
  Synthetic polypeptide
HACDQPISFFHIALSDLTPTDNNCLTASFPFFSNST

SEQ ID NO: 6 - protein, 2 aa, Artificial Sequence
  Synthetic polypeptide
ER

SEQ ID NO: 7 - protein, 83 aa, Artificial Sequence
  Synthetic polypeptide
SSIQLVTVTQKHKLPSEQKHSHNRLLSNLGVSSNSTNPSCKSSCPVFSIALFDSNISCFEKCDKPSIPQTLLFNSFEQCSIQI

SEQ ID NO: 8 - protein, 1 aa, Artificial Sequence
  Synthetic polypeptide
H

SEQ ID NO: 9 - protein, 2 aa, Artificial Sequence
  Synthetic polypeptide
IL

SEQ ID NO: 10 - protein, 37 aa, Artificial Sequence
  Synthetic polypeptide
AFFHKKLELYIDYQLSYFFQIIPYPLFFNEFYHIIAY

SEQ ID NO: 11 - protein, 13 aa, Artificial Sequence
  Synthetic polypeptide
FGIFFLKFDTNTG

SEQ ID NO: 12 - protein, 23 aa, Artificial Sequence
  Synthetic polypeptide
YMLFSHKEYRMLLRLATLLFSNG

SEQ ID NO: 13 - protein, 8 aa, Artificial Sequence
  Synthetic polypeptide
NIFENWKT

SEQ ID NO: 14 - protein, 4 aa, Artificial Sequence
  Synthetic polypeptide
TAYE

SEQ ID NO: 15 - protein, 3 aa, Artificial Sequence
  Synthetic polypeptide
VQK

SEQ ID NO: 16 - protein, 23 aa, Artificial Sequence
  Synthetic polypeptide
IWQPHYSFDYHFTKNKNRVDELF

SEQ ID NO: 17 - protein, 22 aa, Artificial Sequence
  Synthetic polypeptide
TGQKRILKMSNSLNWNITNYAY

SEQ ID NO: 18 - protein, 15 aa, Artificial Sequence
  Synthetic polypeptide
NLMKSYADKMELQLI

SEQ ID NO: 19 - protein, 16 aa, Artificial Sequence
  Synthetic polypeptide
RLCIKDTIFQKNYVHI

SEQ ID NO: 20 - protein, 43 aa, Artificial Sequence
  Synthetic polypeptide
IRGILSRKRVQNLYYRVMLEIIKYLQFFKILCNLPNLSLKILS

SEQ ID NO: 21 - protein, 1 aa, Artificial Sequence
  Synthetic polypeptide
C

SEQ ID NO: 22 - protein, 18 aa, Artificial Sequence
  Synthetic polypeptide
LSTNNLFPMIYMFTFLAF

SEQ ID NO: 23 - protein, 32 aa, Artificial Sequence
  Synthetic polypeptide
FLFEHYNCSKLHQCSKFLRISHFQKFGNLPQL

SEQ ID NO: 24 - protein, 18 aa, Artificial Sequence
  Synthetic polypeptide
LIFSKTFRKIKNVLGWIV

SEQ ID NO: 25 - protein, 7 aa, Artificial Sequence
  Synthetic polypeptide
KFGLVRL

SEQ ID NO: 26 - protein, 13 aa, Artificial Sequence
  Synthetic polypeptide
KYLDYIPTDNASD

SEQ ID NO: 27 - protein, 11 aa, Artificial Sequence
  Synthetic polypeptide
ILLLISSFYAS

SEQ ID NO: 28 - protein, 33 aa, Artificial Sequence
  Synthetic polypeptide
AQKYEKKSSIKKKLEMISNNILRAFFNLGMCWK

SEQ ID NO: 29 - protein, 20 aa, Artificial Sequence
  Synthetic polypeptide
DSIKSSKKSQGTFLKFVSFH

SEQ ID NO: 30 - protein, 11 aa, Artificial Sequence
  Synthetic polypeptide
NTIFSLNSKFS

SEQ ID NO: 31 - protein, 59 aa, Artificial Sequence
  Synthetic polypeptide
VFTEYSTKSTIYSYQIAVITILHFQRTKLAEYRLPLEHFTNSKFIDMRVISQFHFSTSL

SEQ ID NO: 32 - protein, 31 aa, Artificial Sequence
  Synthetic polypeptide
VTSHPLIIIVLLLHFHFSQIPHRSVRVPFNW

SEQ ID NO: 33 - protein, 19 aa, Artificial Sequence
  Synthetic polypeptide
QSHKNTNFPLNKNIVIIVC

SEQ ID NO: 34 - protein, 22 aa, Artificial Sequence
  Synthetic polypeptide
VISVYRQIQPTHPVNPPVLSFQ

SEQ ID NO: 35 - protein, 50 aa, Artificial Sequence
  Synthetic polypeptide
LFLTVTFHVLKNVINLRSPKHFFSILLNNVQYKFNIESYKLFFTKNLSYI

SEQ ID NO: 36 - protein, 9 aa, Artificial Sequence
  Synthetic polypeptide
IINFLISFK

SEQ ID NO: 37 - protein, 22 aa, Artificial Sequence
  Synthetic polypeptide
SLIHYFSMNFIISLLTDLAFSS

SEQ ID NO: 38 - protein, 80 aa, Artificial Sequence
  Synthetic polypeptide
NSTPTLGNTCCSRTKSIVCCFDSPHCFSQMDETFSKIGKPKPHTSECKNKFGNHTTASIITSQKIKTGSTNFFELAKNVF

SEQ ID NO: 39 - protein, 5 aa, Artificial Sequence
  Synthetic polypeptide
KCQTL

SEQ ID NO: 40 - protein, 11 aa, Artificial Sequence
  Synthetic polypeptide
IGISQITHTEI

SEQ ID NO: 41 - protein, 30 aa, Artificial Sequence
  Synthetic polypeptide
RAMPIKWNYNLFDACVLKTQYFKKITFTFR

SEQ ID NO: 42 - protein, 15 aa, Artificial Sequence
  Synthetic polypeptide
GVSSVENVFKTSTIE

SEQ ID NO: 43 - protein, 21 aa, Artificial Sequence
  Synthetic polypeptide
CWKLLNIYNFSKYSVIYQTFH

SEQ ID NO: 44 - protein, 15 aa, Artificial Sequence
  Synthetic polypeptide
KFYHNVNCRPTICFQ

SEQ ID NO: 45 - protein, 6 aa, Artificial Sequence
  Synthetic polypeptide
FICLHF

SEQ ID NO: 46 - protein, 37 aa, Artificial Sequence
  Synthetic polypeptide
RFDNSYLNITIVQNYINARNSLEFRIFRNLAIYHNCD

SEQ ID NO: 47 - protein, 15 aa, Artificial Sequence
  Synthetic polypeptide
FFQKHFEKSKMFWVG

SEQ ID NO: 48 - protein, 12 aa, Artificial Sequence
  Synthetic polypeptide
FKNLVLLDYKNI

SEQ ID NO: 49 - protein, 14 aa, Artificial Sequence
  Synthetic polypeptide
TIFPLTMLQIKSCY

SEQ ID NO: 50 - protein, 21 aa, Artificial Sequence
  Synthetic polypeptide
FLLFTLLKHKNTKKNRQLKKN

SEQ ID NO: 51 - protein, 1 aa, Artificial Sequence
  Synthetic polypeptide
K

SEQ ID NO: 52 - protein, 5 aa, Artificial Sequence
  Synthetic polypeptide
FLIIY

SEQ ID NO: 53 - protein, 25 aa, Artificial Sequence
  Synthetic polypeptide
ERFLILVCVGNEIQSNLQKNHREHF

SEQ ID NO: 54 - protein, 12 aa, Artificial Sequence
  Synthetic polypeptide
SLFHFTETLFFH

SEQ ID NO: 55 - protein, 5 aa, Artificial Sequence
  Synthetic polypeptide
IASFQ

SEQ ID NO: 56 - protein, 18 aa, Artificial Sequence
  Synthetic polypeptide
KFSQSIRQKAQSIPIKLQ

SEQ ID NO: 57 - protein, 29 aa, Artificial Sequence
  Synthetic polypeptide
QFCISNAQSWRNTVFLLNTSRIQNSLTCV

SEQ ID NO: 58 - protein, 11 aa, Artificial Sequence
  Synthetic polypeptide
SANFIFPHRFE

SEQ ID NO: 59 - protein, 4 aa, Artificial Sequence
  Synthetic polypeptide
PHTH

SEQ ID NO: 60 - protein, 33 aa, Artificial Sequence
  Synthetic polypeptide
LSYCFISIFLKFHIGALEFHSTGNSHTKTQTSL

SEQ ID NO: 61 - protein, 3 aa, Artificial Sequence
  Synthetic polypeptide
TKT

SEQ ID NO: 62 - protein, 1 aa, Artificial Sequence
  Synthetic polypeptide
S

SEQ ID NO: 63 - protein, 4 aa, Artificial Sequence
  Synthetic polypeptide
SFAE

SEQ ID NO: 64 - protein, 12 aa, Artificial Sequence
  Synthetic polypeptide
SRCIVKFNQPIL

SEQ ID NO: 65 - protein, 11 aa, Artificial Sequence
  Synthetic polypeptide
ILLSCLFNSSF

SEQ ID NO: 66 - protein, 1 aa, Artificial Sequence
  Synthetic polypeptide
Q

SEQ ID NO: 67 - protein, 4 aa, Artificial Sequence
  Synthetic polypeptide
HFMF

SEQ ID NO: 68 - protein, 2 aa, Artificial Sequence
  Synthetic polypeptide
KM

SEQ ID NO: 69 - protein, 12 aa, Artificial Sequence
  Synthetic polypeptide
TFDPPNTSFQFF

SEQ ID NO: 70 - protein, 19 aa, Artificial Sequence
  Synthetic polypeptide
TMFNTNLTLNLISFFSQKT

SEQ ID NO: 71 - protein, 21 aa, Artificial Sequence
  Synthetic polypeptide
VIYRLSTFLFLSNNPLSIIFQ

SEQ ID NO: 72 - protein, 79 aa, Artificial Sequence
  Synthetic polypeptide
ILSYHCLLIWHFLLEIRHQHWVIHVVLAQRVSYVASTRHTAFLKWMKHFRKLENLNRIRVSAKINLATTLQLRLSLHKK

SEQ ID NO: 73 - protein, 39 aa, Artificial Sequence
  Synthetic polypeptide
KQGRRTFLNWPKTYSKNVKLFKLEYHKLRILKFNEELCR

SEQ ID NO: 74 - protein, 11 aa, Artificial Sequence
  Synthetic polypeptide
NGTTTYLTLVY

SEQ ID NO: 75 - protein, 18 aa, Artificial Sequence
  Synthetic polypeptide
RHNISKKLRSHLDKGYPQ

SEQ ID NO: 76 - protein, 8 aa, Artificial Sequence
  Synthetic polypeptide
KTCSKPLL

SEQ ID NO: 77 - protein, 6 aa, Artificial Sequence
  Synthetic polypeptide
SNVGNY

SEQ ID NO: 78 - protein, 9 aa, Artificial Sequence
  Synthetic polypeptide
IFTIFQNTL

SEQ ID NO: 79 - protein, 36 aa, Artificial Sequence
  Synthetic polypeptide
FTKPFTENFIIMLIVDQQFVSNDLYVYIFSVLIIPI

SEQ ID NO: 80 - protein, 14 aa, Artificial Sequence
  Synthetic polypeptide
TLQLFKTTSMLEIP

SEQ ID NO: 81 - protein, 38 aa, Artificial Sequence
  Synthetic polypeptide
NFAFSEIWQFTTTVIDFFKNISKNQKCFGLDSLKIWSC

SEQ ID NO: 82 - protein, 10 aa, Artificial Sequence
  Synthetic polypeptide
TIKIFRLYSH

SEQ ID NO: 83 - protein, 27 aa, Artificial Sequence
  Synthetic polypeptide
QCFRLNLVINFFFLRFLSTKIRKKIVN

SEQ ID NO: 84 - protein, 7 aa, Artificial Sequence
  Synthetic polypeptide
KKIRNDF

SEQ ID NO: 85 - protein, 6 aa, Artificial Sequence
  Synthetic polypeptide
YTKSVF

SEQ ID NO: 86 - protein, 35 aa, Artificial Sequence
  Synthetic polypeptide
SWYVLEMRFNQIFKKITGNIFEVCFISLKHYFFIE

SEQ ID NO: 87 - protein, 3 aa, Artificial Sequence
  Synthetic polypeptide
QVF

SEQ ID NO: 88 - DNA, 1705 bp, Caenorhabditis elegans
ctgaaaacttgctattcaatgaaaaaatagtgtttcagtgaaatgaaacaaacttcaaaaatgttccctgtgatttttttgaagatttgattgaatctcatttccaacacataccaagattaaaaaacgctcttagtatattattagaaatcatttctaatttttttttaattgacgattttttttcgtatttttgtgcttaagaagcgtaaaaagaagaaattaataacaagatttaatctgaagcattgtcagtgggaatatagtctaaatatttttatagtctaacaagaccaaatttttaaactatccaacccaaaacatttttgatttttcgaaatgtttttgaaaaaatcaatcacagttgtggtaaattgccaaatttctgaaaatgcgaaattctaaggaatttcgagcattgatgtagttttgaacaattgtaatgttcaaataggaattatcaaaacgctaaaaatgtaaacatataaatcattggaaacaaattgttggtcgacaattaacattatgataaaattttcagtgaaaggtttggtaaattacagagtattttgaaaaattgtaaatatttaataatttccaacattactctatagtagaggttttgaacacgttttctactgaggataccccttatctaaatgtgaacgtaatttttttgaaatattgtgtctttaatacacaagcgtcaaataagttgtagttccattttatcggcatagctcttcattaaatttcagtatgcgtaatttgtgatattccaatttaaagagtttgacatttttagaatacgtttttggccagttcaaaaaagttcgtcgaccctgtttttattttttgtgaagtgataatcgaagctgtagtgtggttgccaaatttatttttgcactcactcgtatgcggtttaggttttccaattttcgaaaatgtttcatccatttgagaaaagcagtgtggcgagtcgaagcaacatacgatactctttgtgcgagaacaacatgtattacccagtgttggtgtcgaatttcaagaagaaaatgccaaatcagtaagcaatgatatgataaaattcattgaaaaataatggataagggattatttgaaagaaataagaaagttgataatctatatataactcaagttttttgtgaaaaaaagcttataagattcaatgttaaatttgtattgaacattgttcaaaagaattgaaaagaagtgtttgggggatcgaaggtttatcacatttttcaaaacatgaaatgttactgtcaaaaagagctattgaaaagacaggacaggaggatttacaggatgggttggttgaatttgacgatacaccgagattactcagcaaacgattatgactatgtttttgttcagagggaagtttgtgtttttgtgtgactgttaccagttgaatggaactctaacgctcctatgtggaatttgagaaaaatggaaatgaagcagtaagacaattattatcagtgggtgtgaggtcactcaaagcgatgtggaaaaatgaaattggctgatcacacgcatgtcaatgaattttgaattcgtgaagtgttcaagaggaagacggtattccgccagctttgtgcgttggaaatgcaaaattgttatcactgcaatttgataggaatagattgtgctttttgtcgaatactctgtgaaaacttac

SEQ ID NO: 89 - protein, 9 aa, Artificial Sequence
  Synthetic polypeptide
LKTCYSMKK

SEQ ID NO: 90 - protein, 19 aa, Artificial Sequence
  Synthetic polypeptide
CFSEMKQTSKMFPVIFLKI

SEQ ID NO: 91 - protein, 10 aa, Artificial Sequence
  Synthetic polypeptide
LNLISNTYQD

SEQ ID NO: 92 - protein, 7 aa, Artificial Sequence
  Synthetic polypeptide
KTLLVYY

SEQ ID NO: 93 - protein, 7 aa, Artificial Sequence
  Synthetic polypeptide
KSFLIFF

SEQ ID NO: 94 - protein, 38 aa, Artificial Sequence
  Synthetic polypeptide
LTIFFRIFVLKKRKKKKLITRFNLKHCQWEYSLNIFIV

SEQ ID NO: 95 - protein, 13 aa, Artificial Sequence
  Synthetic polypeptide
QDQIFKLSNPKHF

SEQ ID NO: 96 - protein, 24 aa, Artificial Sequence
  Synthetic polypeptide
FFEMFLKKSITVVVNCQISENAKF

SEQ ID NO: 97 - protein, 24 aa, Artificial Sequence
  Synthetic polypeptide
GISSIDVVLNNCNVQIGIIKTLKM

SEQ ID NO: 98 - protein, 30 aa, Artificial Sequence
  Synthetic polypeptide
TYKSLETNCWSTINIMIKFSVKGLVNYRVF

SEQ ID NO: 99 - protein, 5 aa, Artificial Sequence
  Synthetic polypeptide
KIVNI

SEQ ID NO: 100 - protein, 15 aa, Artificial Sequence
  Synthetic polypeptide
FPTLLYSRGFEHVFY

SEQ ID NO: 101 - protein, 17 aa, Artificial Sequence
  Synthetic polypeptide
GYPLSKCERNFFEILCL

SEQ ID NO: 102 - protein, 5 aa, Artificial Sequence
  Synthetic polypeptide
YTSVK

SEQ ID NO: 103 - protein, 19 aa, Artificial Sequence
  Synthetic polypeptide
VVVPFYRHSSSLNFSMRNL

SEQ ID NO: 104 - protein, 28 aa, Artificial Sequence
  Synthetic polypeptide
YSNLKSLTFLEYVFGQFKKVRRPCFYFL

SEQ ID NO: 105 - protein, 71 aa, Artificial Sequence
  Synthetic polypeptide
SDNRSCSVVAKFIFALTRMRFRFSNFRKCFIHLRKAVWRVEATYDTLCARTTCITQCWCRISRRKCQISKQ

SEQ ID NO: 106 - protein, 5 aa, Artificial Sequence
  Synthetic polypeptide
YDKIH

SEQ ID NO: 107 - protein, 43 aa, Artificial Sequence
  Synthetic polypeptide
KIMDKGLFERNKKVDNLYITQVFCEKKLIRFNVKFVLNIVQKN

SEQ ID NO: 108 - protein, 64 aa, Artificial Sequence
  Synthetic polypeptide
KEVFGGSKVYHIFQNMKCYCQKELLKRQDRRIYRMGWLNLTIHRDYSANDYDYVFVQREVCVFV

SEQ ID NO: 109 - protein, 22 aa, Artificial Sequence
  Synthetic polypeptide
LLPVEWNSNAPMWNLRKMEMKQ

SEQ ID NO: 110 - protein, 7 aa, Artificial Sequence
  Synthetic polypeptide
DNYYQWV

SEQ ID NO: 111 - protein, 20 aa, Artificial Sequence
  Synthetic polypeptide
GHSKRCGKMKLADHTHVNEF

SEQ ID NO: 112 - protein, 42 aa, Artificial Sequence
  Synthetic polypeptide
IREVFKRKTVFRQLCALEMQNCYHCNLIGIDCAFCRILCENL

SEQ ID NO: 113 - protein, 5 aa, Artificial Sequence
  Synthetic polypeptide
KLAIQ

SEQ ID NO: 114 - protein, 7 aa, Artificial Sequence
  Synthetic polypeptide
KNSVSVK

SEQ ID NO: 115 - protein, 8 aa, Artificial Sequence
  Synthetic polypeptide
NKLQKCSL

SEQ ID NO: 116 - protein, 2 aa, Artificial Sequence
  Synthetic polypeptide
FF

SEQ ID NO: 117 - protein, 3 aa, Artificial Sequence
  Synthetic polypeptide
RFD

SEQ ID NO: 118 - protein, 13 aa, Artificial Sequence
  Synthetic polypeptide
ISFPTHTKIKKRS

SEQ ID NO: 119 - protein, 7 aa, Artificial Sequence
  Synthetic polypeptide
YIIRNHF

SEQ ID NO: 120 - protein, 4 aa, Artificial Sequence
  Synthetic polypeptide
FFFN

SEQ ID NO: 121 - protein, 16 aa, Artificial Sequence
  Synthetic polypeptide
RFFFVFLCLRSVKRRN

SEQ ID NO: 122 - protein, 4 aa, Artificial Sequence
  Synthetic polypeptide
QDLI

SEQ ID NO: 123 - protein, 8 aa, Artificial Sequence
  Synthetic polypeptide
SIVSGNIV

SEQ ID NO: 124 - protein, 3 aa, Artificial Sequence
  Synthetic polypeptide
IFL

SEQ ID NO: 125 - protein, 21 aa, Artificial Sequence
  Synthetic polypeptide
SNKTKFLNYPTQNIFDFSKCF

SEQ ID NO: 126 - protein, 7 aa, Artificial Sequence
  Synthetic polypeptide
KNQSQLW

SEQ ID NO: 127 - protein, 17 aa, Artificial Sequence
  Synthetic polypeptide
IAKFLKMRNSKEFRALM

SEQ ID NO: 128 - protein, 1 aa, Artificial Sequence
  Synthetic polypeptide
F

SEQ ID NO: 129 - protein, 6 aa, Artificial Sequence
  Synthetic polypeptide
TIVMFK

SEQ ID NO: 130 - protein, 5 aa, Artificial Sequence
  Synthetic polypeptide
ELSKR

SEQ ID NO: 131 - protein, 18 aa, Artificial Sequence
  Synthetic polypeptide
KCKHINHWKQIVGRQLTL

SEQ ID NO: 132 - protein, 3 aa, Artificial Sequence
  Synthetic polypeptide
NFQ

SEQ ID NO: 133 - protein, 3 aa, Artificial Sequence
  Synthetic polypeptide
KVW

SEQ ID NO: 134 - protein, 8 aa, Artificial Sequence
  Synthetic polypeptide
ITEYFEKL

SEQ ID NO: 135 - protein, 45 aa, Artificial Sequence
  Synthetic polypeptide
IFNNFQHYSIVEVLNTFSTEDTPYLNVNVIFLKYCVFNTQASNKL

SEQ ID NO: 136 - protein, 9 aa, Artificial Sequence
  Synthetic polypeptide
FHFIGIALH

SEQ ID NO: 137 - protein, 11 aa, Artificial Sequence
  Synthetic polypeptide
ISVCVICDIPI

SEQ ID NO: 138 - protein, 2 aa, Artificial Sequence
  Synthetic polypeptide
RV

SEQ ID NO: 139 - protein, 2 aa, Artificial Sequence
  Synthetic polypeptide
HF

SEQ ID NO: 140 - protein, 51 aa, Artificial Sequence
  Synthetic polypeptide
NTFLASSKKFVDPVFIFCEVIIEAVVWLPNLFLHSLVCGLGFPIFENVSSI

SEQ ID NO: 141 - protein, 46 aa, Artificial Sequence
  Synthetic polypeptide
EKQCGESKQHTILFVREQHVLPSVGVEFQEENAKSVSNDMIKFIEK

SEQ ID NO: 142 - protein, 16 aa, Artificial Sequence
  Synthetic polypeptide
WIRDYLKEIRKLIIYI

SEQ ID NO: 143 - protein, 9 aa, Artificial Sequence
  Synthetic polypeptide
LKFFVKKSL

SEQ ID NO: 144 - protein, 7 aa, Artificial Sequence
  Synthetic polypeptide
DSMLNLY

SEQ ID NO: 145 - protein, 22 aa, Artificial Sequence
  Synthetic polypeptide
TLFKRIEKKCLGDRRFITFFKT

SEQ ID NO: 146 - protein, 8 aa, Artificial Sequence
  Synthetic polypeptide
NVTVKKSY

SEQ ID NO: 147 - protein, 12 aa, Artificial Sequence
  Synthetic polypeptide
KDRTGGFTGWVG

SEQ ID NO: 148 - protein, 1 aa, Artificial Sequence
  Synthetic polypeptide
I

SEQ ID NO: 149 - protein, 39 aa, Artificial Sequence
  Synthetic polypeptide
RYTEITQQTIMTMFLFRGKFVFLCDCYQLNGTLTLLCGI

SEQ ID NO: 150 - protein, 4 aa, Artificial Sequence
  Synthetic polypeptide
EKWK

SEQ ID NO: 151 - protein, 19 aa, Artificial Sequence
  Synthetic polypeptide
SSKTIIISGCEVTQSDVEK

SEQ ID NO: 152 - protein, 38 aa, Artificial Sequence
  Synthetic polypeptide
NWLITRMSMNFEFVKCSRGRRYSASFVRWKCKIVITAI

SEQ ID NO: 153 - protein, 1 aa, Artificial Sequence
  Synthetic polypeptide
E

SEQ ID NO: 154 - protein, 12 aa, Artificial Sequence
  Synthetic polypeptide
IVLFVEYSVKTY

SEQ ID NO: 155 - protein, 12 aa, Artificial Sequence
  Synthetic polypeptide
ENLLFNEKIVFQ

SEQ ID NO: 156 - protein, 53 aa, Artificial Sequence
  Synthetic polypeptide
NETNFKNVPCDFFEDLIESHFQHIPRLKNALSILLEIISNFFLIDDFFSYFCA

SEQ ID NO: 157 - protein, 2 aa, Artificial Sequence
  Synthetic polypeptide
EA

SEQ ID NO: 158 - protein, 8 aa, Artificial Sequence
  Synthetic polypeptide
KEEINNKI

SEQ ID NO: 159 - protein, 8 aa, Artificial Sequence
  Synthetic polypeptide
SEALSVGI

SEQ ID NO: 160 - protein, 12 aa, Artificial Sequence
  Synthetic polypeptide
SKYFYSLTRPNF

SEQ ID NO: 161 - protein, 27 aa, Artificial Sequence
  Synthetic polypeptide
TIQPKTFLIFRNVFEKINHSCGKLPNF

SEQ ID NO: 162 - protein, 10 aa, Artificial Sequence
  Synthetic polypeptide
KCEILRNFEH

SEQ ID NO: 163 - protein, 6 aa, Artificial Sequence
  Synthetic polypeptide
CSFEQL

SEQ ID NO: 164 - protein, 14 aa, Artificial Sequence
  Synthetic polypeptide
CSNRNYQNAKNVNI

SEQ ID NO: 165 - protein, 10 aa, Artificial Sequence
  Synthetic polypeptide
IIGNKLLVDN

SEQ ID NO: 166 - protein, 30 aa, Artificial Sequence
  Synthetic polypeptide
HYDKIFSERFGKLQSILKNCKYLIISNITL

SEQ ID NO: 167 - protein, 2 aa, Artificial Sequence
  Synthetic polypeptide
RF

SEQ ID NO: 168 - protein, 10 aa, Artificial Sequence
  Synthetic polypeptide
TRFLLRIPLI

SEQ ID NO: 169 - protein, 1 aa, Artificial Sequence
  Synthetic polypeptide
M

SEQ ID NO: 170 - protein, 1 aa, Artificial Sequence
  Synthetic polypeptide
T

SEQ ID NO: 171 - protein, 2 aa, Artificial Sequence
  Synthetic polypeptide
FF

SEQ ID NO: 172 - protein, 19 aa, Artificial Sequence
  Synthetic polypeptide
NIVSLIHKRQISCSSILSA

SEQ ID NO: 173 - protein, 8 aa, Artificial Sequence
  Synthetic polypeptide
LFIKFQYA

SEQ ID NO: 174 - protein, 32 aa, Artificial Sequence
  Synthetic polypeptide
FVIFQFKEFDIFRIRFWPVQKSSSTLFLFFVK

SEQ ID NO: 175 - protein, 3 aa, Artificial Sequence
  Synthetic polypeptide
SKL

SEQ ID NO: 176 - protein, 14 aa, Artificial Sequence
  Synthetic polypeptide
CGCQIYFCTHSYAV

SEQ ID NO: 177 - protein, 47 aa, Artificial Sequence
  Synthetic polypeptide
VFQFSKMFHPFEKSSVASRSNIRYSLCENNMYYPVLVSNFKKKMPNQ

SEQ ID NO: 178 - protein, 3 aa, Artificial Sequence
  Synthetic polypeptide
AMI

SEQ ID NO: 179 - protein, 7 aa, Artificial Sequence
  Synthetic polypeptide
NSLKNNG

SEQ ID NO: 180 - protein, 3 aa, Artificial Sequence
  Synthetic polypeptide
GII

SEQ ID NO: 181 - protein, 2 aa, Artificial Sequence
  Synthetic polypeptide
KK

SEQ ID NO: 182 - protein, 2 aa, Artificial Sequence
  Synthetic polypeptide
ES

SEQ ID NO: 183 - protein, 8 aa, Artificial Sequence
  Synthetic polypeptide
SIYNSSFL

SEQ ID NO: 184 - protein, 8 aa, Artificial Sequence
  Synthetic polypeptide
KKAYKIQC

SEQ ID NO: 185 - protein, 61 aa, Artificial Sequence
  Synthetic polypeptide
ICIEHCSKELKRSVWGIEGLSHFSKHEMLLSKRAIEKTGQEDLQDGLVEFDDTPRLLSKRL

SEQ ID NO: 186 - protein, 17 aa, Artificial Sequence
  Synthetic polypeptide
LCFCSEGSLCFCVTVTS

SEQ ID NO: 187 - protein, 3 aa, Artificial Sequence
  Synthetic polypeptide
MEL

SEQ ID NO: 188 - protein, 34 aa, Artificial Sequence
  Synthetic polypeptide
RSYVEFEKNGNEAVRQLLSVGVRSLKAMWKNEIG

SEQ ID NO: 189 - protein, 5 aa, Artificial Sequence
  Synthetic polypeptide
SHACQ

SEQ ID NO: 190 - protein, 4 aa, Artificial Sequence
  Synthetic polypeptide
ILNS

SEQ ID NO: 191 - protein, 36 aa, Artificial Sequence
  Synthetic polypeptide
SVQEEDGIPPALCVGNAKLLSLQFDRNRLCFLSNTL

SEQ ID NO: 192 - protein, 2 aa, Artificial Sequence
  Synthetic polypeptide
KL

SEQ ID NO: 193 - DNA, 1518 bp, Caenorhabditis elegans
ccataacgattgaaaacagccgcaaacggaagatggacggcccgaagccgaacctggcgtcggccgcttcgatggagtcgttgaacagtgtttcttctgaagcaacgaatccatcacaggtccgcaccttgtttgtttcgggtcttccaatggatgctaagccgcgtgagctttatcttctgttccgtggatgtcgtggttatgagggagctcttttgaaaatgacatcgaagaatggaaaaccaacgtctccagtcggatttgtcacctttctttcgcaacaagatgcgcaggacgccagaaaaatgttgcaaggtgtccgattcgatccggaatgtgcacaggtacttcgactagaacttgcaaaatcgaacacaaaagtagctcgacctaaacaatctccaccaccaccacaacatgcggcactatcagccgctgcagccggagtcccgcagtttttggcaccaatgcaacacgatcttctactagatcctcaatcagcagctcttttcaatgagcaacaactattggctctttcacttccacatttacatgctgcacaggcacttcaagcagcctatatgccagcttctgctctacaacaatacagtcagaatcaattgtttgcagctgctcaaatgcacccagcagccgctgcagcagccagcctccaacattctcaacaagcatctcaagcttccacctctgcgtgctccactcttttcgtcgccaatctatcggctgaagtgaatgaagatactcttcggggtgtattcaaagcattctctggtttcacacgtctacgactgcacaacaagaatggatcatgtgtggcgtttgttgaatattcggatcttcagaaagcaactcaagcgatgatatcactacaaggattccagataacagccaacgatcgaggtggtcttcgtattgaatatgctcggaacaagatggctgatgtgaatggataagaagagaagcaagaattctttgcagttttgttttgggaagcgcaccaccatcaccaaatgcacaatcatcactcagcaactactacttctacttcttcttcttcttcctgtgcacaaccaccaccacctccaccaacgtacacccgctctcattacacttagtgcattttcgtccttttttcattttactctcccaaaaaatcaccaaaaatcctttcggatctctttttattcgcattttattttcctttctcttaatttttacaaaattcgaagtgtttgtaagcaccctacaggaacttctattgtcttgaccgtagagctcctttgcatcatatcttttctatttctgatttactctctgtaaatatatatcacgagagcttatccacctgtctgtctgtccgaacctgaaattttctgtgatttgttctaatttttcataagatcctttttccccttttaccaccaatcatcatgcattccagtctctcttctgttgtaatttgaaaaagcttttgtaataaatttatacactttattggtt

SEQ ID NO: 194 - protein, 312 aa, Caenorhabditis elegans
MDGPKPNLASAASMESLNSVSSEATNPSQVRTLFVSGLPMDAKPRELYLLFRGCRGYEGALLKMTSKNGKPTSPVGFVTFLSQQDAQDARKMLQGVRFDPECAQVLRLELAKSNTKVARPKQSPPPPQHAALSAAAAGVPQFLAPMQHDLLLDPQSAALFNEQQLLALSLPHLHAAQALQAAYMPASALQQYSQNQLFAAAQMHPAAAAAASLQHSQQASQASTSACSTLFVANLSAEVNEDTLRGVFKAFSGFTRLRLHNKNGSCVAFVEYSDLQKATQAMISLQGFQITANDRGGLRIEYARNKMADVNG

SEQ ID NO: 195 - DNA, 26 bp, Artificial Sequence
  Synthetic oligonucleotide
gatccaaaaatggatccaacgaatta

SEQ ID NO: 196 - DNA, 26 bp, Artificial Sequence
  Synthetic oligonucleotide
ggggttgcggatccaagcagtttgaa

(19) United States
(12) Patent Application Publication
(10) Pub. No.: US 2009/0137046 A1
(43) Pub. Date: May 28, 2009

(54) Splicing-Mediated Regulation Of Gene Expression

(76) Inventors: Andrea Calixto, New York, NY (US); Charles Ma, Palo Alto, CA (US); Martin Chalfie, New York, NY (US)

Correspondence Address: BAKER BOTTS L.L.P., 30 ROCKEFELLER PLAZA, 44TH FLOOR, NEW YORK, NY, 10112-4498, US

(21) Appl. No.: 11/931,166

(22) Filed: Oct. 31, 2007

Related U.S. Application Data
(63) Continuation of application No. PCT/US06/16968, filed on May 2, 2006.
(60) Provisional application No. 60/677,132, filed on May 2, 2005.

(51) Int. Cl. C12N 15/87; C07H 21/00; C12N 15/63; C12N 5/10

(52) U.S. Cl. 435/463; 536/23.1; 435/320.1; 435/325

(57) ABSTRACT

The present invention relates to methods and compositions for controlling the expression of a target gene, whereby an intron cassette such as INT9, an intronic mec-2-derived element, is incorporated into the target gene and expression of the product of the target gene is conditional upon functional expression of the RNA processing protein, mec-8.

### CROSS-REFERENCE TO RELATED APPLICATION

This application claims priority to U.S. Provisional Application Ser. No. 60/677,132 filed May 2, 2005, which is hereby incorporated by reference in its entirety herein.

### GRANT INFORMATION

The subject matter of this application was developed at least in part under National Institutes of Health Grant No. GM30997, so that the United States Government has certain rights herein.

### 1. INTRODUCTION

The present invention relates to methods and compositions for controlling the expression of a target gene, whereby an intron cassette such as INT9, an intronic mec-2-derived element, is incorporated into the target gene and expression of the product of the target gene is conditional upon functional expression of the RNA processing protein, MEC-8.

### 2. BACKGROUND OF THE INVENTION

### 2.1 Control of Gene Expression

Whether the goal has been to study the function of a gene, or to conditionally produce a gene with known effect, scientists have attempted, for many years, to find ways to effectively control expression of a gene of interest (Meyer-Ficca et al., 2004, Anal. Biochem. 334(1):9-19). A number of imperfect solutions have been found, typically in the form of inducible promoters, such as tetracycline-responsive Tet systems (Tet-On, Tet-Off; Gopalkrishnan et al., 1999, Nucleic Acids Res. 27(24):4775-4782); the glucocorticoid-responsive mouse mammary tumor virus promoter (MMTVprom) inducible with dexamethasone (Israel and Kaufman, 1989, Nucleic Acids Res. 17(12):4589-4604) and the ecdysone-inducible promoter (EcP) (No et al., 1996, Proc. Natl. Acad. Sci. U. S. A. 93(8):3346-3351). Typically, however, the inducible promoters depend upon the presence of an exogenously added “trigger” molecule, which potentially perturbs the cell or organism being studied from its natural condition. It is therefore desirable to develop a means of conditionally controlling gene expression which does not depend on exposure to an exogenous triggering agent. 2.2 MEC-2

Touch sensitivity in animals relies on nerve endings in the skin that convert mechanical force into electrical signals. The response to gentle touch in the nematode Caenorhabditis elegans is mediated by a set of six mechanosensory receptor neurons (Gu et al., 1996, Proc. Natl. Acad. Sci. U. S. A. 93(13):6577-6582) that express two amiloride-sensitive Na⁺ channel proteins. Saturation mutageneses for touch-insensitive animals have led to the identification of 13 genes (called “mec” for MEChanosensory abnormal) that are needed for the function of these touch receptors. Mutant animals are touch insensitive (the Mec phenotype) but have fully differentiated touch receptor neurons. Mechanosensory touch cells are comprised of touch cell-specific microtubules mec-12 and mec-7 (corresponding to α-tubulin and β-tubulin, respectively). Microtubule displacement leads to channel opening and translation of physical contact to the mechanosensory stimulus of sensory neurons (Huang et al., 1995, Nature 378(6554):292-295). Mechanosensation requires the degenerin channel complex, which contains four proteins, MEC-2, MEC-4, MEC-6 and MEC-10 (Zhang et al., 2004, Curr. Biol. 14(21)1888-1896). Thus, the mec-2 gene product is involved in transducing signals generated by application of an external force.

### 2.3 MEC-8

Mutations in the mec-8 gene of C. elegans have been shown to affect the functions of body wall muscle and mechanosensory and chemosensory neurons (Chalfie and Au, 1989, Science 243(4894 Pt 1):1027-1033). The original temperature sensitive mutant of mec-8 (u218 ts) is heat sensitive and the mutant gene product is inactive when the growth temperature is shifted from the permissive temperature of 15° C. to the non-permissive temperature, 25° C. (Chalfie and Au 1989 Science 243(4894 Pt 1):1027-1033). This mutation was found to cause defective touch cell function. An additional eight mec-8 mutants (Lundquist and Herman, 1994, Genetics 138:83-101) were shown to result in disruptions in the structure of body wall muscle. Analysis showed that mutations in mec-8 strongly enhanced the mutant phenotype of specific mutations in the gene, unc-52. unc-52 encodes, via alternative splicing of its pre-mRNA, a set of basement membrane proteins, homologs of perlecan, that are important for body wall muscle assembly and attachment to basement membrane, hypodermis and cuticle (Lundquist and Herman, 1994, Genetics 138:83-101).

The cloned mec-8 gene product was found to encode a protein with two RNA recognition motifs, characteristic of RNA binding proteins (Lundquist and Herman, 1994, Genetics 138:83-101). Experiments have shown that mec-8 regulates the accumulation of a specific subset of alternatively spliced unc-52 transcripts. Utilizing antibodies to UNC-52 it has been shown that MEC-8 affects the abundance of a subset of UNC-52 isoforms. Thus mec-8 was demonstrated to encode a trans-acting factor that regulates the alternative splicing of the pre-mRNA of unc-52 and one or more additional genes that affect mechanosensory and chemosensory responses (Lundquist et al., 1996, Development 122: 1601-1610).

More recent work has shown that MEC-8 is a nuclear protein found in the hypodermis at most stages of development and not in most late embryonic or larval body-wall muscle, and thus may be a long-lived, highly stable protein. Use of tissue-specific unc-52 minigene expression constructs fused to green fluorescent protein allowed monitoring of tissue-specific mec-8-dependent alternative splicing of unc-52 mRNA. From these studies it was shown that mec-8 had to be expressed in the same cell as the unc-52 minigene in order to regulate its expression, supporting the view that MEC-8 acted directly on unc-52 transcripts (Spike et al., 2002, Development 129(21):4999-5008) to regulate the alternative splicing of the pre-mRNA of unc-52.

### 3. SUMMARY OF THE INVENTION

The present invention relates to methods and compositions which enable the regulation of expression of a gene of interest by conditional splicing. It is based, at least in part, on the discoveries that (i) an intronic sequence derived from the C. elegans mec-2 gene, when inserted in a target gene, renders expression of the target gene conditional on the expression of a second C. elegans gene, mec-8, (ii) a temperature sensitive mutant of mec-8 allowed expression of the target gene to be turned on by switching from the non-permissive to the permissive temperature, (iii) repeated cycles of induced expression of the target gene may be achieved by cyclic provision of the inducer, and (iv) temperature sensitive splicing of a molecule required for RNAi function could be used to control expression of a gene of interest.

Thus, the present invention provides methods and materials for controlling gene expression, whereby expression of diverse genes can be rendered conditional on splicing and/or temperature sensitive. Furthermore, suppression of gene expression by RNAi can be transformed into a conditional event.

### 4. BRIEF DESCRIPTION OF THE FIGURES

FIG. 1. Sequence of the mec-2 intron 9 (“INT9”; lowercase, nucleotides 84-1788) with flanking exonic DNA (uppercase); (SEQ ID NO:1). The consensus “GT-AG” splice boundary nucleotides are underlined. The entire sequences of exons 9 and 10 which flank intron 9 on either side are also shown.

FIG. 2A-B. (A) Sequence analysis of the mec-2 INT9 sequence (SEQ ID NO:2) for presence of stop codons in three forward and reverse reading frames. The * symbol indicates the position of a stop codon in either the forward three reading frames (Direct Translation) or in the reverse frames (Antiparallel translation). Nucleotide position 1 of this sequence corresponds to nucleotide number 84 of SEQ ID NO:1 (FIG. 1). The amino acid sequence of the first reading frame (directly below the INT9 sequence) is SEQ ID NO:3; the amino acid sequence of the second reading frame (directly below the amino acid sequence of the first reading frame) is SEQ ID NO:4; and the amino acid sequence of the third reading frame (directly below the amino acid sequence of the second reading frame) is SEQ ID NO:5.

(B) Sequence analysis of anti-parallel INT9 sequence (SEQ ID NO:6), with three possible anti-parallel translation reading frames (SEQ ID NOS: 7, 8, and 9, respectively, sequentially numbered as in (A) above).

FIG. 3A-B. (A) Sequence of the mec-8 gene (GenBank Accession No. NM_{—}060107; SEQ ID NO: 10) showing the complete open reading frame (ORF) from nucleotide numbers 33 to 971 and additional flanking sequences.

(B) Amino acid sequence of MEC-8 (SEQ ID NO:11).

FIG. 4A-B. Schematic showing that mec-2 mRNA processing requires mec-8.

(A) mec-2 in wild type C. elegans. (B) mec-2 in C. elegans lacking mec-8 (“mec-8(0)).

FIG. 5A-G. Including mec-2 INT9 in a reporter construct confers MEC-8 dependence, (A) Reporter construct P_{mec-18}intron 9::yfp showing the mec-18 promoter driving expression of a YFP fusion construct comprising INT9. (B) When P_{mec-18}intron 9::yfp is introduced into C. elegans in the absence of active MEC-8 (animals having an inactive mutation, mec-8 (u314)), no YFP is detectable. (C) When P_{mec-18}intron 9::yfp is introduced into C. elegans in the presence of active MEC-8, YFP is detectable. (D) Little or no fluorescence from YFP in mec-8 (u314) mutant worms carrying the construct. Left panel is phase interference image; right panel is fluorescence microscopy image. (E) Fluorescence from YFP expressed in touch receptor neurons in an animal containing P_{mec-18}intron 9::yfp and functional MEC-8. (F). Little or no fluorescence from YFP in touch receptor neurons of C. elegans containing P_{mec-18}intron 9::yfp but having a temperature sensitive mutation in mec-8 (mec-8 (u218ts)) at the non-permissive temperature (25° C.). (G) Fluorescence from YFP in touch receptor neurons of C. elegans containing P_{mec-18}intron 9::yfp and a temperature sensitive mutation in mec-8 (mec-8 (u218ts)) at the permissive temperature (15° C.).

FIG. 6A-D. In C. elegans having a temperature sensitive mutation in mec-8 (mec-8 (u218ts)) as well as the construct P_{mec-4}intron 9:mec-4, mec-4 expression was essentially temperature sensitive. (A) P_{mec-4}intron 9:mec-4 construct. (B) Expression of an endogenous temperature sensitive mec-4 mutant, mec-4(u45)ts. (C) Expression of temperature sensitive mutant mec-8 (mec-8 (u218ts)). (D) Expression of P_{mec-4}intron 9:mec-4 in C. elegans lacking active MEC-4, where mec-8 is temperature sensitive (mec-8 (u218ts)).

FIG. 7A-B. Using temperature sensitive mec-8 and an INT9-rde-1 construct to make RNAi function temperature sensitive, where RDE-1 is required for RNAi function. (A) Construct P_{rde-1}intron 9:rde-1. (B) RNAi sensitivity in C. elegans containing P_{rde-1}intron 9:rde-1, in the presence or absence of active MEC-8, in certain instances where the mec-8 allele is temperature sensitive at the non-permissive (25° C.) or permissive (15° C.) temperature.

### 5. DETAILED DESCRIPTION OF THE INVENTION

The present invention relates to a system for controlling expression of a target gene, comprising an intron cassette such as INT9 (sequence of intron 9 of the mec-2 gene as set forth in GenBank Accession No. U26736) inserted into the target gene, and a MEC-8 protein that is conditionally functional. The system operates in the context of a cell, which may or may not be part of a multicellular organism. Preferably, the system operates in a C. elegans cell, but it is envisaged that the invention may be applied to other organisms.

For clarity, and not by way of limitation, the detailed description of the invention is divided into the following subsections:
(i) intron cassettes;
(ii) target genes;
(iii) mec-8;
(iv) intron cassette/target gene constructs;
(v) intron cassette/target gene/mec8 gene expression control systems; and
(vi) uses of the invention.

### 5.1 Intron Cassettes

The present invention provides for the use of an intron cassette (“IC”), excisable by wild-type or otherwise functional MEC-8 (lower case italic letters denote the gene, capital unitalicized letters denote the protein). In a preferred, non-limiting embodiment, the intron cassette is INT9, but the invention envisages the use of other MEC-8 excisable sequences as well, such as the sequences excised by MEC-8-dependent splicing of exon 15 to exon 19 or exon 16 to exon 19 of unc-52 (Spike et al., 2002, Development 129(21):4999-5008). The disclosure herein applied to INT9 may be analogously applied to such other intronic sequences.

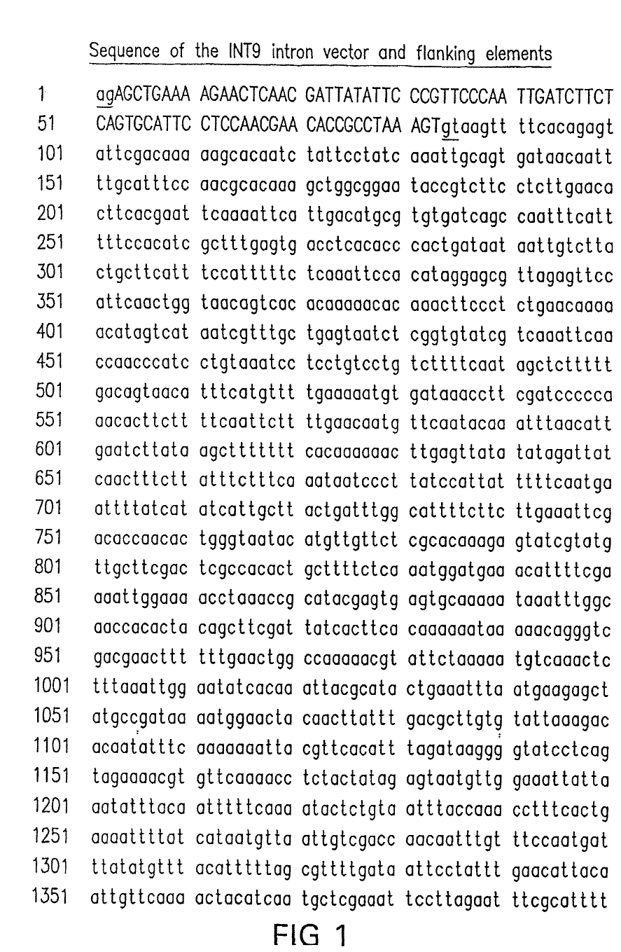

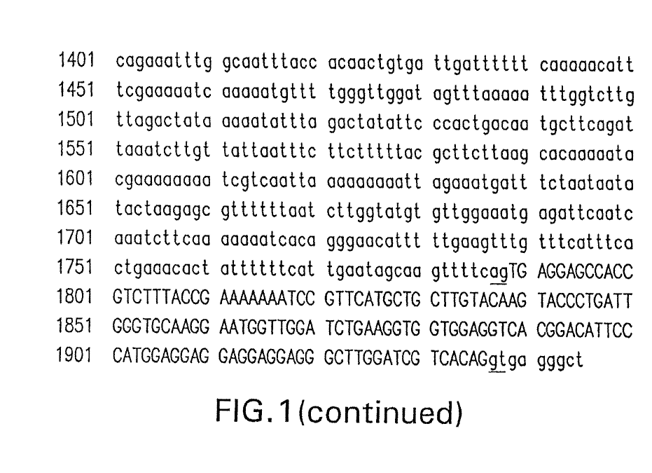

The present invention provides for an INT9 sequence, which is derived from the 9^{th} intron of the C. elegans mec-2 gene and several adjacent nucleotides of exon sequence. Preferably, INT9 is comprised in the sequence set forth in FIG. 1 (SEQ ID NO:1) between residues 84 and 1788 (one specific non-limiting example of INT9 sequence is SEQ ID NO:2). The term “INT9,” as used herein, further applies to (i) nucleic acid molecules comprising portions of the sequence set forth in FIG. 1 (SEQ ID NO:1) between residues 84 and 1788 (SEQ ID NO:2) which, when comprised in a target gene, may be excised by MEC-8; (ii) nucleic acid molecules which are at least 100, 200, 300, 400, 500, 600, 700, 800, 900, 1000, 1100, 1200, 1300, 1400, 1500, 1600 or 1700 nucleotides in length and which hybridize to the sequence as set forth in FIG. 1 (SEQ ID NO:1) between residues 84 and 1788 under stringent conditions (defined herein as hybridization in 0.5 M NaHPO4, 7 percent sodium dodecyl sulfate (“SDS”), 1 mM ethylenediamine tetraacetic acid (“EDTA”) at 65° C., and washing in 0.1× SSC/0.1 percent SDS at 68° C. (Ausubel et al., 1989, Current Protocols in Molecular Biology, Vol. I, Green Publishing Associates, Inc., and John Wiley & Sons, Inc. New York, at p. 2.10.3); (iii) nucleic acid molecules which are, as a result of nucleotides that are added, deleted, or substituted, at least 80, 85, 90, or 95 percent homologous to the sequence set forth in FIG. 1 between residues 84 and 1788 (SEQ ID NO:2), as determined by standard software using BLAST, FASTA or other sequence similarity search algorithms; and (iv) nucleic acid molecules which comprise at least 50, at least 100, or at least 200 consecutive non-exon nucleotides from both the 5′ and 3′ ends of the INT9 sequence set forth in FIG. 1 between residues 84 and 1788, and nucleic acid molecules that are at least 80, 85, 90 or 95 percent homologous thereto.

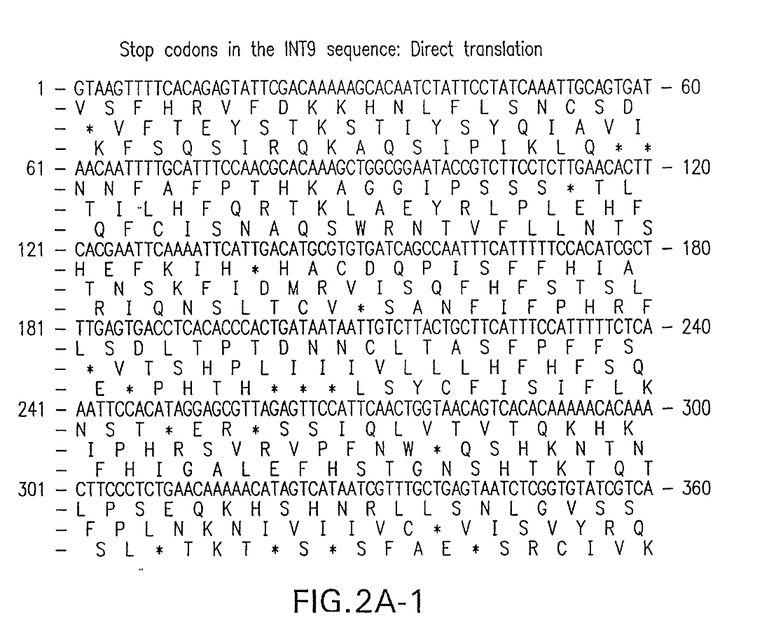

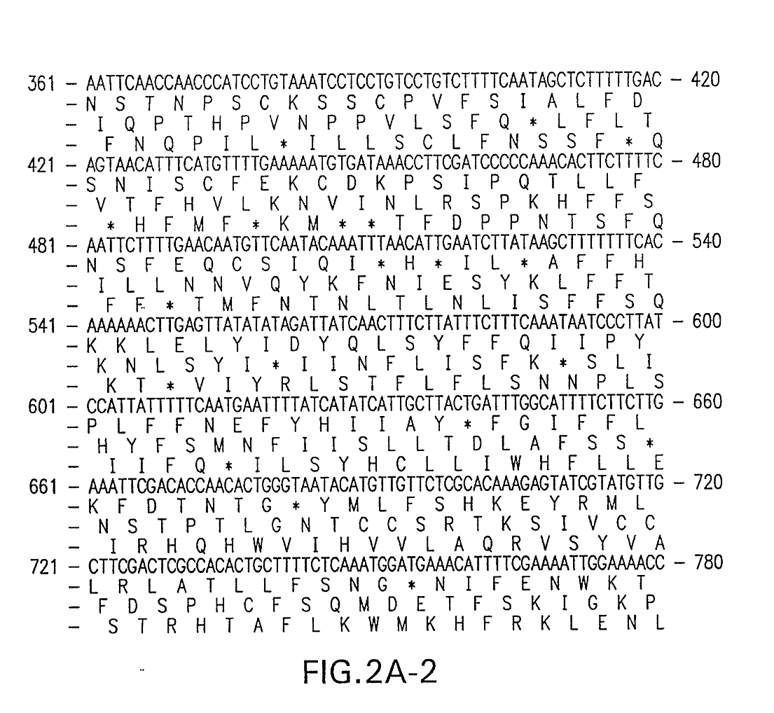

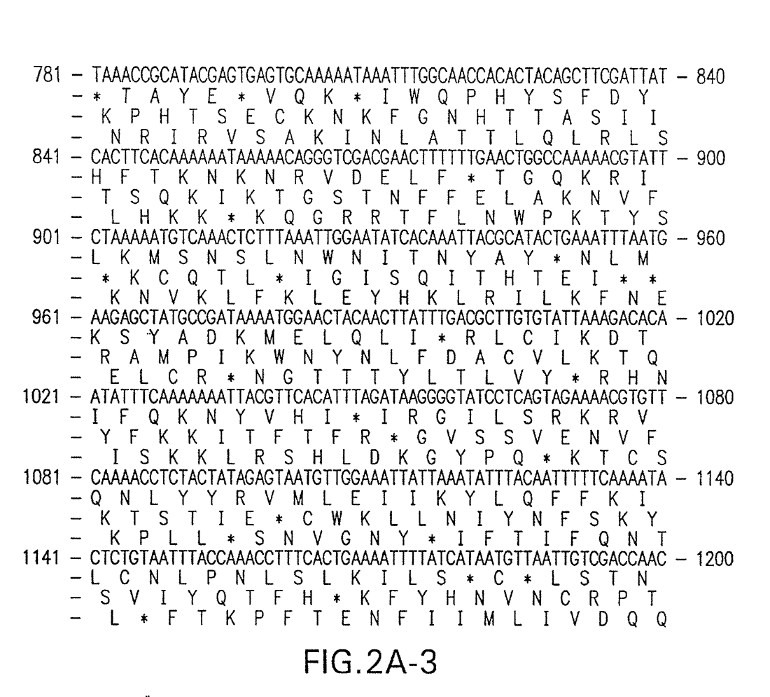

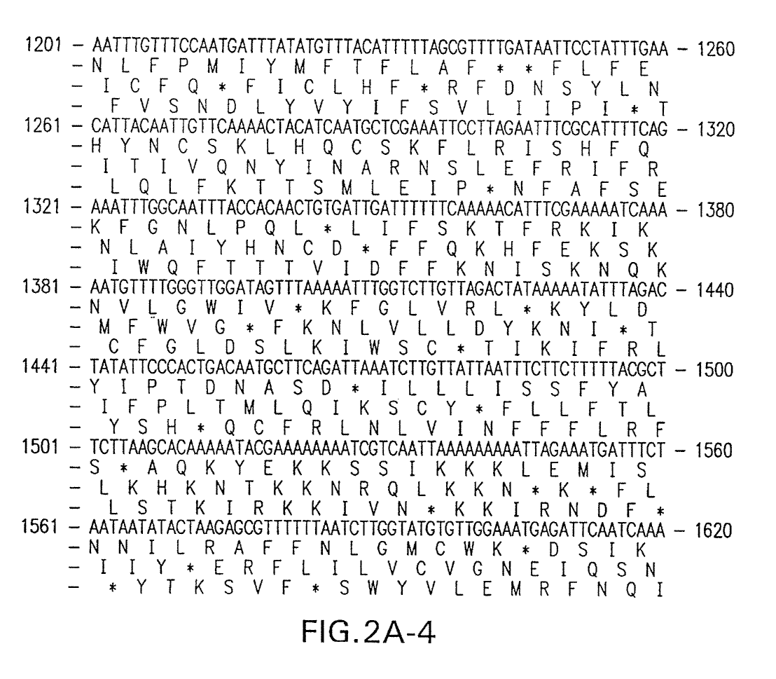

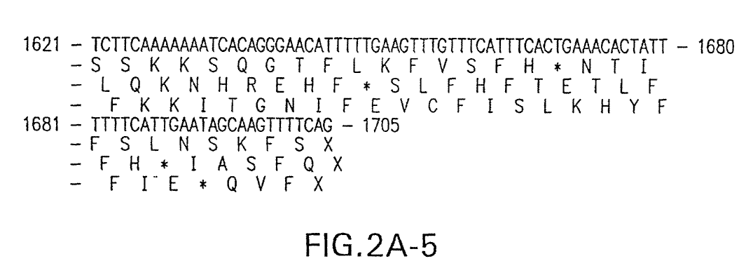

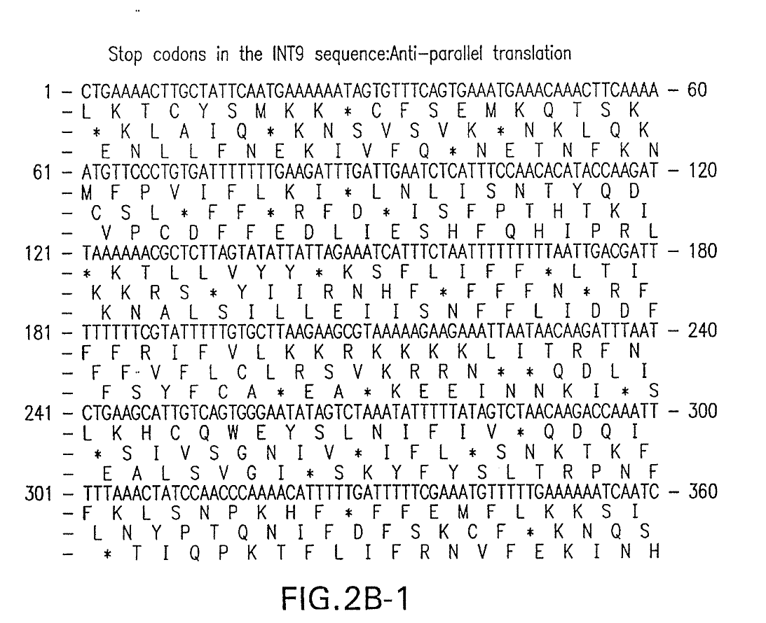

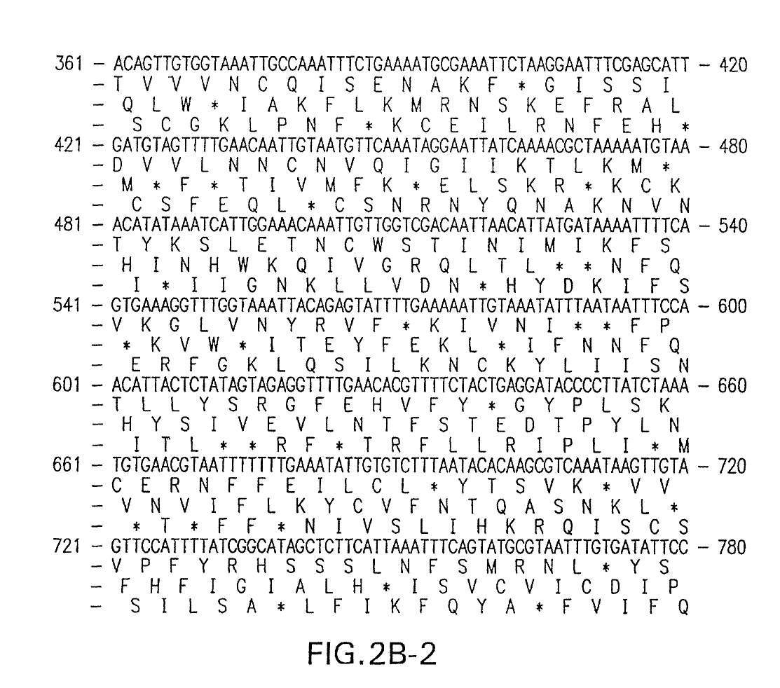

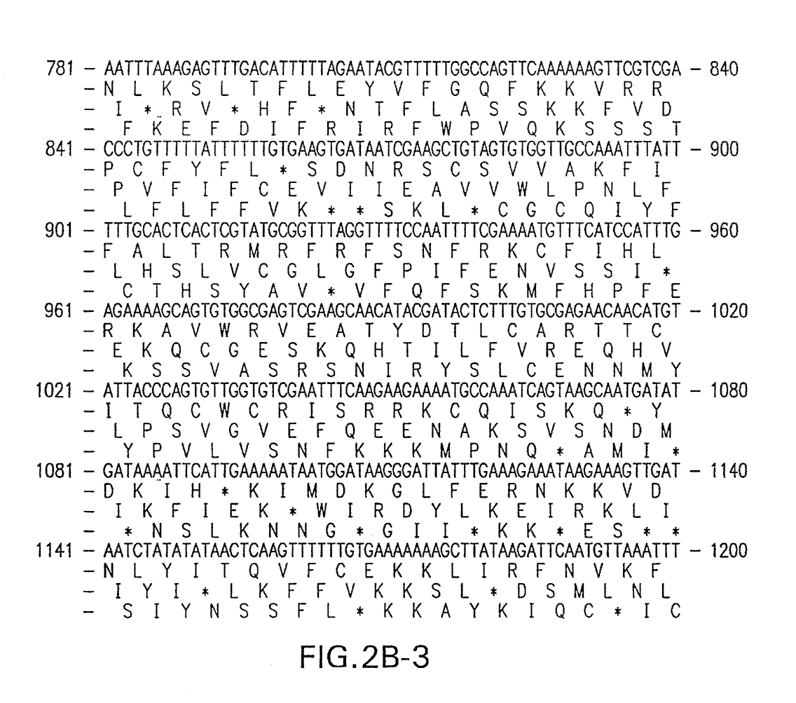

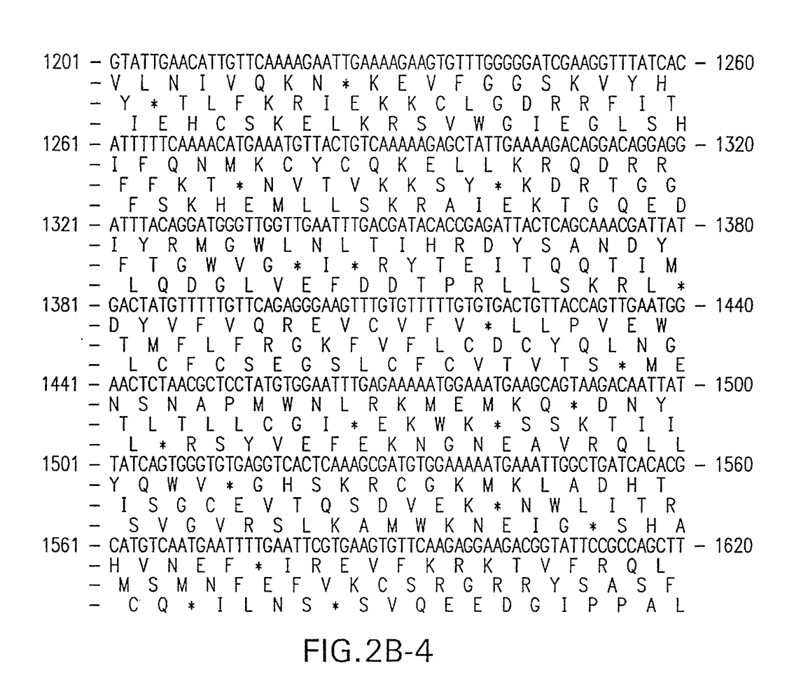

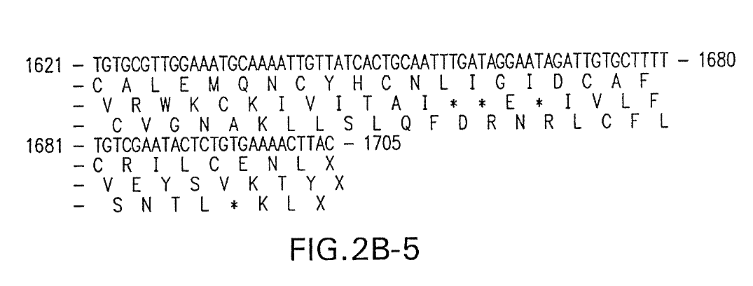

The INT9 sequence between nucleotide residues 84 and 1788 of SEQ ID NO:1 (FIG. 1) contains several short protein coding open reading frames interrupted by translational stop codons in all forward and reverse (“anti-parallel”) frames (SEQ ID NO: 2; FIG. 2A-B). Therefore the likelihood of an artificial, inadvertent or undesirable protein expressed following insertion or replacement of the INT9 sequence into a heterologous gene as provided by the invention is not likely to occur by “readthrough” irrespective of the reading frame of the targeted sequence.

In non-limiting embodiments of the invention, an IC may be modified so as to supplement its ability to block target gene expression. For example, the IC may be modified to introduce one or more translational stop-codon(s) in a specific reading frame in order to avoid “read-through” translation of a partially spliced or unspliced mRNA. For example, the inserted stop codon may be chosen from any of the three known translational stop sequences “TAA”, “TAG” or “TGA”. The invention also provides for the insertion, into the IC, of a small oligonucleotide cassette which contains a stop codon on all three forward and/or all three reverse frames. Design, synthesis and insertion of an appropriate stop-codon oligonucleotide can be performed using standard laboratory methods.

In other non-limiting embodiments, the present invention provides for the inclusion of the 5′ “GT” and 3′ “AG” splice consensus signals at either extremity of the IC sequence and optionally additional mec-2 derived or exogenous nucleotides may be added to the 5′ and 3′ ends of the IC sequence to facilitate insertion into the target sequence or enhance excision by MEC-8. According to the invention, insertion of any additional flanking sequences should, after excision of the IC, maintain the reading frame of the interrupted target gene sequence so that a functional gene product may be expressed

The IC may be inserted into an appropriate plasmid vector so that it may be easily propagated and maintained, and so that the integrity and stability of the IC sequence is not compromised by inadvertent mutation or recombination during propagation. For example, the plasmid vector may have flanking polylinker sequences, oligonucleotide primer binding sites or other recognition sequences for enzymes such as site specific recombinases that facilitate IC insertion into a target gene.

### 5.2 Target Genes

Virtually any gene may be a target according to the invention. While in preferred embodiments the target gene encodes a protein product, the present invention may also be applied to RNA products, for example RNAi, where the insertion of an IC would disrupt function.

Accordingly, as non-limiting examples, the target gene may be a gene that encodes an ion channel, a tumor suppressor protein, an oncogenic protein, a toxic protein, a protein involved in signal transduction, such as a kinase or a phosphatase, a protein that promotes apoptosis, a receptor protein, a growth factor or other cytokine, a hormone, etc.

The target gene may be a gene of any organism, including but not limited to an insect such as Drosophila melanogaster, a worm such as Caenorhabditis elegans, an amphibian such as Xenopus laevis, a protozoan such as Plasmodium falciparum or Trypanosoma cruzi, a fish such as Danio rerio, a bird such as Gallus gallus, a rodent such as Rattus rattus or Mus musculus, or a caprine, bovine, ovine, porcine or primate species, including Homo sapiens. In addition, the target gene may be a gene of virus.

In specific, non-limiting embodiments, the target gene may be rde-1 or rde-4 (Parrish and Fire, 2001, RNA 7:1397-1402) or another gene which is necessary for RNA interference in C. elegans. Analogous genes related to RNAi activity in other species may further be used as target genes, including members of the Dicer and Argonaute (PAZ domain proteins; Yan et al., 2004, Nature 426(6965):486-474) gene family in plants and animals. In additional embodiments components of the RISC complex isolated from D. melanogaster, C. elegans, and human may be targeted, including mammalian and Drosophila AGO2 proteins, mammalian GEMIN3 (a DEAD box helicase) and GEMIN4 proteins, Drosophila dFXR (a homologue of the human fragile X mental retardation protein) etc.

### 5.3 MEC-8

In one set of non-limiting embodiments, the present invention utilizes a conditionally functional MEC-8 protein. The term “mec-8 gene” encompasses wild type and mutant mec-8 alleles. “Functional” means that MEC-8 is able to efficiently excise an intron excisable by wild-type MEC-8 under the same conditions. “Efficiently” means at least 50 percent, at least 60 percent, at least 70 percent, at least 80 percent, or at least 90 percent relative to wild-type enzyme. “Conditional” means that under non-permissive conditions, the efficiency decreases by at least about 30 percent, at least about 40 percent, at least about 50 percent, at least about 60 percent, at least about 70 percent, at least about 80 percent, or at least about 90 percent.

In other non-limiting embodiments, the present invention provides for the use of non-conditionally (constitutively) functional MEC-8 protein where protein expression is conditional, either by virtue of conditional transcription (see below), conditional transport of mRNA out of the nucleus, conditional translation (e.g. RNAi controlled), or other factors.

In still other embodiments, introduction of a nucleic acid encoding non-conditionally functional MEC-8 protein may be a trigger that activates INT9 splicing and expression of a gene of interest.

In yet further embodiments, the present invention provides for the use of conditionally expressed, conditionally functional MEC-8.

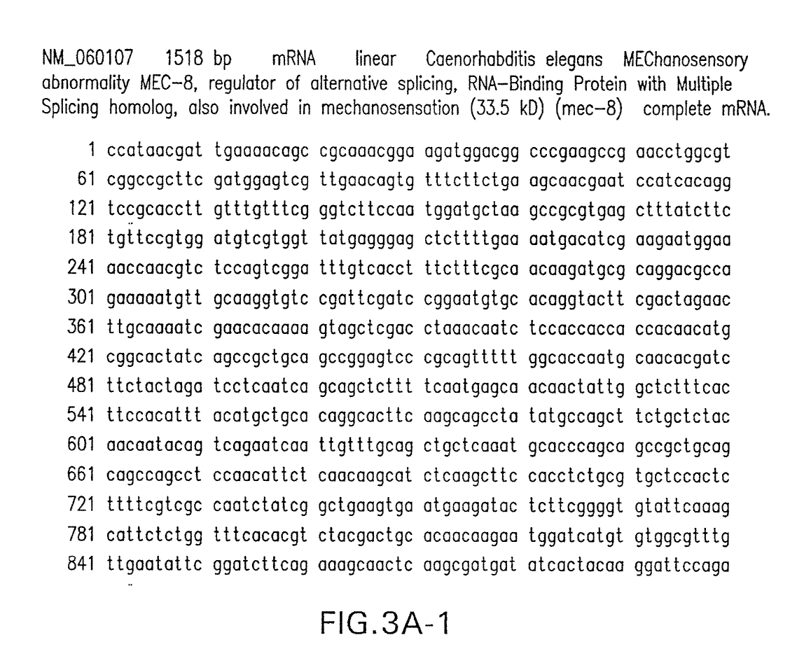

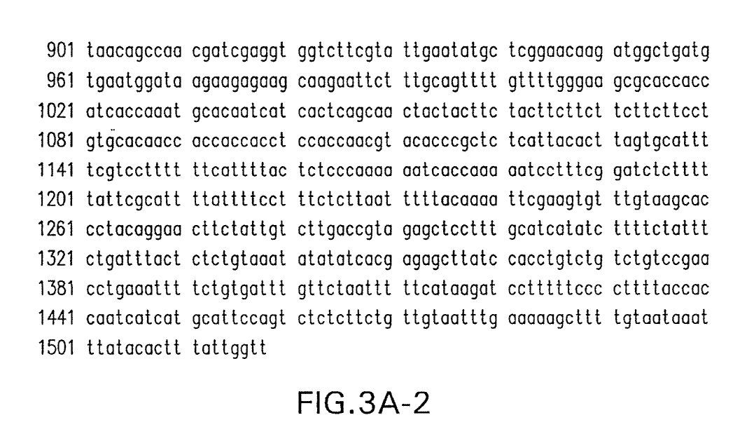

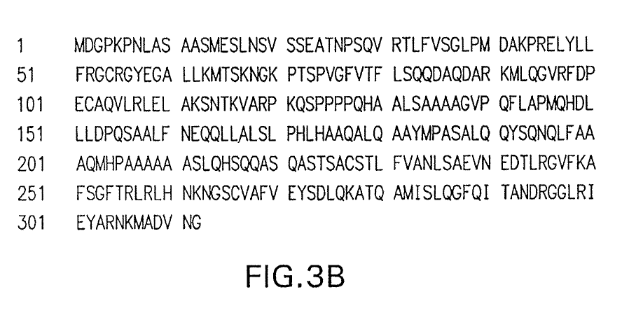

The nucleic acid sequence encoding wild type MEC-8, as well as the amino acid sequence of wild type MEC-8 protein, are set forth in FIGS. 3A and 3B, respectively. In a preferred specific embodiment of the invention, the temperature sensitive mutant of MEC-8 is exemplified by the mec-8 u218 ts allele (Chalfie and Au, 1989, Science 243(4894 Pt 1):1027-1033). This first reported temperature sensitive mutant of mec-8 is heat sensitive so that the mutant gene product is inactive when the growth temperature is shifted from 15° C. to 25° C. (Chalfie and Au, 1989, Science 243(4894 Pt l):1027-1033). The molecular nature of the mec-8 u218 ts allele is a conserved amino acid change of an alanine residue at position 278 (codon GCA) to a threonine residue (codon ACA).

The invention provides for additional temperature sensitive or additionally modified mec-8 alleles, mutants or fusion genes which encode a MEC-8 protein whose activity may be switched on or off in a cell of interest. Non-limiting embodiments of alternative mec-8 alleles or mutants include but are not limited to a MEC-8 protein which has a shorter half-life than the wild-type protein, which preferably is a variant MEC-8 such as the temperature sensitive mutant encoded by u218 ts, modified to comprise a PEST sequence (Li et al., 1998, J. Biol. Chem. 273:34970-34975; Leclerc et al., 2000, Biotechniques 29:590-598), using the N-end rule (Bachmair et al., 1986, Science 234: 179-186) or creating a cleavable ubiquitin fusion construct (Johnson et al., 1995, J. Biol. Chem. 270:17442-17456). As a specific, non-limiting example, a Praja E3-ubiquitin ligase ring finger domain may be fused to all or a portion of the temperature sensitive MEC-8 mutant encoded by u218ts.

Further examples of mutants of mec-8 which may be used according to the invention include the mutants described in Lundquist and Herman, 1994, Genetics 138:83-101.

Preferably, the conditional nature of MEC-8's functionality is a result of protein structure. However, the present invention also provides for conditional functionality resulting from transcriptional differences. In non-limiting embodiments, the present invention provides for a system in which an endogenous promoter/mec-8 gene is not expressed (e.g., a C. elegans mutant or another type of organism (e.g., Drosophila, human)), but in which mec-8 is operably linked to a promoter which is active during a particular developmental stage or an inducible promoter (e.g., a tetracycline-inducible promoter; a tamoxifen-inducible promoter; such embodiments are less preferred because they utilize an exogenous agent). Thus, splicing of the gene of interest would be controlled by the presence or absence of inducing agent (e.g., tamoxifen or tetracycline). Although such embodiments may use wild-type MEC-8 or an equivalent thereof, in certain non-limiting embodiments of the invention, once turned on, to turn the splicing “off”, a destabilized version of MEC-8 (e.g., a cleavable ubiquitin fusion construct comprising the wild-type MEC-8 or a variant thereof) may be used.

### 5.4 Intron Cassette/Target Gene Constructs

The present invention provides for IC/target gene constructs. The IC may be inserted at any point of the gene, where “gene” refers to that portion of the genomic sequence which is transcribed into RNA. Preferably the target gene is not mec-2. Accordingly, the present invention provides for a nucleic acid comprising an intron cassette/target gene construct comprising a target gene which is not mec-2 interrupted by an INT9 sequence inserted into a region of the target gene upstream of the end of its coding sequence, such that retention of INT9 in a mRNA transcript of the construct would interfere with expression of functional target gene product (gene product exhibiting at least about 30, 40, 50, 60, 70, 80 or 90 percent of the activity of the wild type gene product). “Interfere with” in this context means decrease, inhibit, or prevent.

A nucleic acid comprising an IC/Target gene construct may be operably linked to a promoter element, which may or may not be the promoter element endogenously linked to the target gene. Suitable promoters include consitutive promoters, tissue specific promoters, inducible promoters, and any promoter known in the art, where selection of a suitable promoter may depend on the particular objective of the construct.

Where the target gene encodes a protein product, an IC may be inserted into a protein encoding region of the target gene, or into an untranslated region. Greater control over expression may be achieved by inserting the IC into the coding region. To avoid the formation of substantial partial target gene product, the IC is preferably inserted in the 5′ end of the target gene, for example between −100 and +100 nucleotides relative to the “A” of the start codon ATG. One or more than one IC may be inserted into a target gene. Where the target gene encodes an RNA product, the IC may be inserted into a region of the RNA which has functional activity, such as providing complementarity to another nucleic acid sequence, or catalytic activity.

The IC may be inserted into the target gene using any method known in the art. In non-limiting embodiments, the method may be practiced in vitro using standard recombinant DNA methods. For example, oligonucleotide primer sequences flanking the IC sequence may be used for PCR amplification or PCR-mediated insertion of the IC sequence into the target gene.

Alternatively, IC may be inserted into the target gene in vivo using, for example, genetic recombination. For example, and not by way of limitation, an IC-targeting construct, comprising an IC (e.g., INT9) flanked on either side by appropriate regions of the target gene (exon-intron boundary of target gene) may be introduced into a cell such that site-specific homologous recombination which inserts the IC into the target gene occurs (Thomas et al., 1986, Cell 44(3):419-28).

Where insertion of IC into the target gene is performed in vivo in a cell, in specific non-limiting embodiments of the invention, the cell may be used to regenerate an animal. Thus, the invention provides for targeted disruption of a gene in an oocyte or an embryonic stem (ES) cell. Alternatively, the cell may be used to give rise to a homogeneous population of cells in culture.

In still further non-limiting embodiments, the IC sequence may be inserted into the target gene by mediation of site specific recombinases known to the art such as the cre- or flp-enzymes (Tronce et al., 2002, FEBS Lett. 529(l):116-121), either in vitro or in vivo using, for example, transgenic animals.

Where the IC/target gene is comprised in an isolated nucleic acid, said nucleic acid may be comprised in a vector, The vector may be a plasmid, bacteriophage or virus. The IC/target gene may optionally be operably linked to an appropriate promoter element and/or additional element that facilitates expression.

### 5.5 Intron Cassette/MEC-8 Gene Expression Control Systems

The IC/target gene may be introduced into a host cell in which functional MEC-8 is conditionally expressed.

Where IC insertion is effected by homologous recombination in vivo, it would not be necessary to introduce the IC/target gene into the host system. Where the IC/target gene are comprised in an isolated nucleic acid molecule, that isolated nucleic acid molecule, optionally comprised in a vector, may be introduced into a host cell by means known to the art including but not limited to electroporation, transfection, microinjection and ballistic methods or via mediation of a biological delivery agent such as an adenovirus, retrovirus or lentivirus.

In one set of non-limiting embodiments, the host cell is a C. elegans cell in which essentially no wild-type MEC-8 is present (that is to say, there is insufficient amount of wild-type MEC-8 to produce detectable splicing of MEC-2), and where the MEC-8 present is conditionally functional. In specific non-limiting embodiments, the conditionally functional MEC-8 is the temperature sensitive mutant encoded by u218ts.

In further non-limiting embodiments of the invention, a system analogous to that described above for C. elegans may be established in another organism. For example, a Drosophila cell, optionally in the context of an intact organism, may be engineered to contain an IC (e.g., INT9) insertionally inactivated target gene and may further contain a temperature sensitive mec-8 allele such as u218 ts. Shift to a permissive temperature (e.g., about 15° C.) may be predicted to enable the splicing of INT9 sequence from the gene of interest and restoration of gene expression in the Drosophila cell. As another example, a similar system may be generated in a human cell containing a target gene having an IC insertion and stable expression of a temperature sensitive mec-8 allele, whereby switching the cell to a permissive temperature induces expression of the target gene.

### 5.6 Uses of the Invention

An advantage of the present invention is that it may provide “tighter” control of gene expression relative to inducible promoter-based systems. An inducible promoter, even in the absence of inducing agent, may still exhibit a significant baseline activity. In contrast, the presence of an IC such as INT9 destroys the expressibility of the target gene; fortuitous correct excision of the IC, or incomplete excision that would permit functional expression of the target gene, would be extremely unlikely to occur.

In a first set of embodiments, the invention may be used to evaluate the consequences of turning the target gene “on” by creating conditions under which the MEC-8 of the system is functional.

For example, and not by way of limitation, where the target gene is rde-1 and the host system is C. elegans (or, in another organism, an analogous RNAi-associated gene is the target gene), the invention may be used to turn “on” RNA interference, and thereby turn “off” the gene targeted by the RNAi.

In further non-limiting embodiments, the present invention provides tools for analyzing a particular regulatory circuit by indirect targeting of a molecule in the circuit. For example, but not by way of limitation, the p53 tumor suppressor protein level may be regulated in a cell or animal system by inserting an IC such as INT9 into an mdm2 target gene. The level of p53 protein may then be ablated by inducing the expression of functional MEC-8 protein in the cell, which in turn permits expression of MDM2 protein, causing p53 degradation.

In yet another set of non-limiting embodiments, the present invention may be used to identify molecules that interact in a physiologic pathway. For example, regulatable expression of a target gene by the method of the present invention may be used to generate differential gene expression patterns which may be analyzed by microarray or other gene expression profiling methods. Thus, total or poly(A)⁺ mRNA may be isolated from a cell or population of cells under conditions wherein the target gene is in the “off” state and separately from a comparable sample in which the target gene is “on.” A gene expression profiling study may then be performed to determine the effect of induction of the target gene by comparing RNA expression profiles between the two samples.

It should be noted that MEC-8 protein, including temperature sensitive MEC-8 encoded by u218 ts, is a very stable protein, such that when conditions permitting function have once occurred, the resulting functional MEC-8 protein is likely to persist for some time, making it difficult to switch the target gene “off”. It therefore may be desirable to utilize a form of MEC-8 which is engineered to have a shorter half-life, for example, a MEC-8 engineered to be fused to a PEST sequence or a Praja E3-ubiquitin ligase ring finger domain.

### 6. EXAMPLE 1

### 6.1 Materials and Methods

C. elegans growth and strains. Wild-type C. elegans (N2) and strains with mutations in mec-8(u314, e398, or u218 ts)I (Chalfie and Au, 1989; Davies et al., 1999) and/or rde-1(ne219)V (Tabara et al., 1999) were usually grown at 20° C. according to Brenner (1974). For experiments testing temperature sensitivity, animals were tested after growth for several generations at either 15° C. or 25° C.

Expression constructs and transformation. The 1.8 kb sequence that contains intron 9 of mec-2 (“INT9”)with the flanking exons (FIG. 1) was amplified by PCR from (genomic or mec-2 vector) using the following primers that introduced 5′ and 3′ BamHI sites: 5′ GATCCAAAAATGGATCCAACGAATTA 3′ (SEQ ID NO:12) and 5′ GGGGTTGCGGATCCAAGCAGTTTGAA 3′ (SEQ ID NO:13). The resulting PCR product was cut with BamHI and cloned into TU#739 between the mec-18 promoter and the yfp (Yellow Fluorescent Protein, a variant or analog or equivalent of Green Fluorescent Protein) coding sequence P_{mec-18}Intron9::yfp or placed between the rde-1 promoter and the rde-1 genomic coding (P_{rde-1}Intron 9::rde-1) sequence in Fire vector pPD95.75 (www.ciwemb.edu/pages/firelab.html). The insertion of the mec-2 sequences introduced INT9, but no new ATG, so the translation start of the products was not altered.

Transgenic animals were generated by microinjecting 2 to 5 ng/μl of the expression plasmid, 40 ng/μl of pRF4 dominant Roller plasmid with the YFP vector; (Mello et al, 1991) or 20 ng/μl of pCW2.1 (a ceh-22::gfp plasmid; Okkema and Fire, 1994) with the rde-1 plasmid, and pBSK plasmid to a final concentration of 100 ng/μl for the injection mix (Mello et al, 1991). At least 5 stable lines were generated for each injection and all of them behaved in similar manners. Wild type, u314, e398 and u218 were transformed with the YFP plasmid. To further demonstrate the dependency of YFP expression on mec-8, the stable lines obtained from the mec-8 mutants e398 and u314 were crossed with wild-type males and assessed the expression of GFP in the heterozygous F1. The rde-1 vector was transformed into strains that had the rde-1(ne219) mutation and either a wild-type of mutant allele mec-8 (e398, u314 and u218).

Phenotypic Characterization: GFP expression: YFP fluorescence was observed using a Zeiss Axioscope 2 or a Leica dissecting microscope equipped for fluorescence microscopy. Animals were also observed and photographed using the DIC optics to record the presence of the touch receptor neurons when YFP was not present. To study the kinetics of YFP restoration in mec-8(u218) animals, we moved the animals from 25° C. to 15° C. at various times after hatching. The observations were made every 15 minutes for the first four hours after the switch and then every hour for the next few hours.

RNA sensitivity: RNAi responses were tested by growth on bacteria making dsRNA for unc-22, unc-52, or rpl-3 according to the procedure of Timmons and Fire, 1998. For experiments with the mec-8(u218) mutants, synchronized larvae of different ages from animals grown at 25° C. in the presence of freshly induced RNAi bacteria at 15° C. P0 and F1 animals were scored in a blind test for the mutant.

### 6.2 Results and Discussion

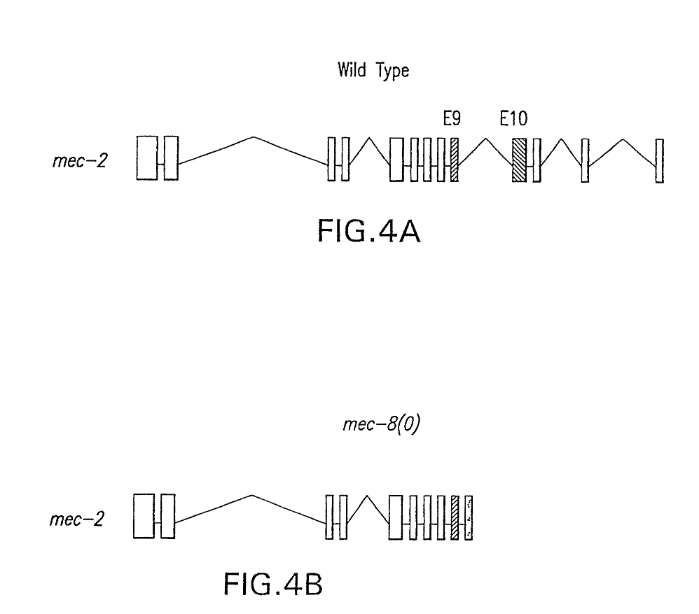

MEC-8 is a nuclear protein that contains two RNA recognition motifs, and is involved in RNA processing [Lundquist et al., 1996, Development 122: 1601-1610]. The initial mec-8 mutations were identified because they produce touch insensitivity, and we have identified mec-2 as a target of mec-8-dependent processing (see FIG. 4A-B). Wild-type animals express two mec-2 mRNAs, mec-2a and mec-2b; mec-2a contains 13 exons and encodes a protein of 481 amino acids. mec-2b is identical to mec-2a through exon 9 followed by an alternative exon contained in intron 9 and a polyA tail. The splicing of mec-2 intron 9 is dependent on mec-8, since mec-2b mRNA, but not mec-2a mRNA, is present in mec-8 animals. mec-2 is not the only gene whose transcript is processed in a mec-8-dependent fashion. Touch insensitivity from a mec-2 null allele, but not from a mec-8 null allele, is rescued by mec-2 genomic DNA lacking intron 9. Because tile rescue was incomplete, although readily apparent, we do not know if mec-2b is important for touch receptor function.

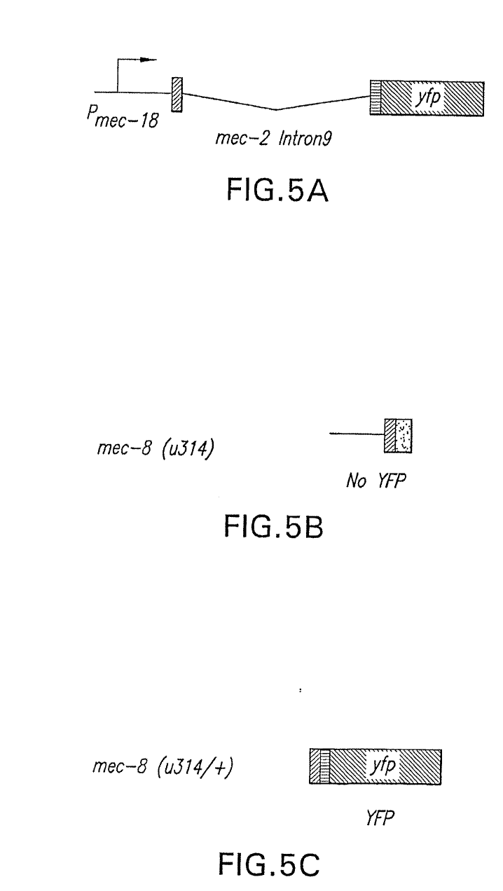

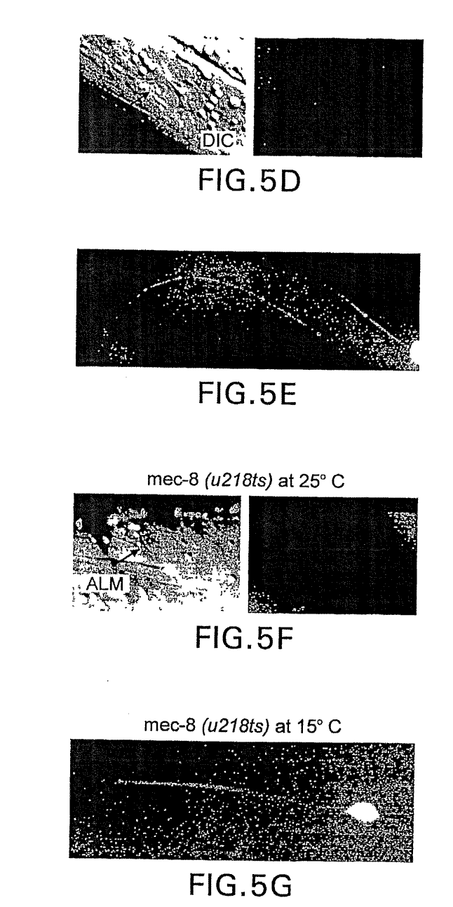

Inclusion of mec-2 intron 9 (INT9) is sufficient to convey mec-8-dependent regulation. We placed INT9 before the YFP in a construct driven ftom the touch cell-specific mec-18 promoter (P_{mec-18}Intron9::yfp; FIG. 5A). No YFP fluorescence was observed in mec-8(e398) or mec-8(u314) (FIGS. 5B and D) animals transformed with P_{mec-18}Intron9::yfp. Fluorescence was seen in all six touch receptor neurons, however, in the progeny of these transgenic animals that have been crossed with wild-type males (FIGS. 5C and E).

The u218 mutation, an Ala278Thr change in the second RRM, results in a temperature-sensitive mec-8 phenotype. mec-8(u218) animals are wild type at 15° C. and touch insensitive at 25° C. It was found that animals transformed with P_{mec-18}intron 9::yfp have fluorescent touch receptor neurons at 15° C., but not at 25° C. (FIGS. 5G and F, respectively). Because mec-8 is expressed in a variety of cells (including several types of neurons and the hypodermis) and is also ubiquitously expressed in the embryo, mec-8 and INT9 may be used to produce temperature-sensitive expression for many genes.

RNA interference (RNAi) has become a very valuable means of reducing gene expression, which would be even more value if RNAi functionality were rendered conditional. To this end, mec-2 INT9 was used to produce functionally temperature-dependent RNAi, based on the fact that the gene rde-1, which encodes the C. elegans homologue of Argonaute2, is required for RNAi (Tabara et al., 1999).

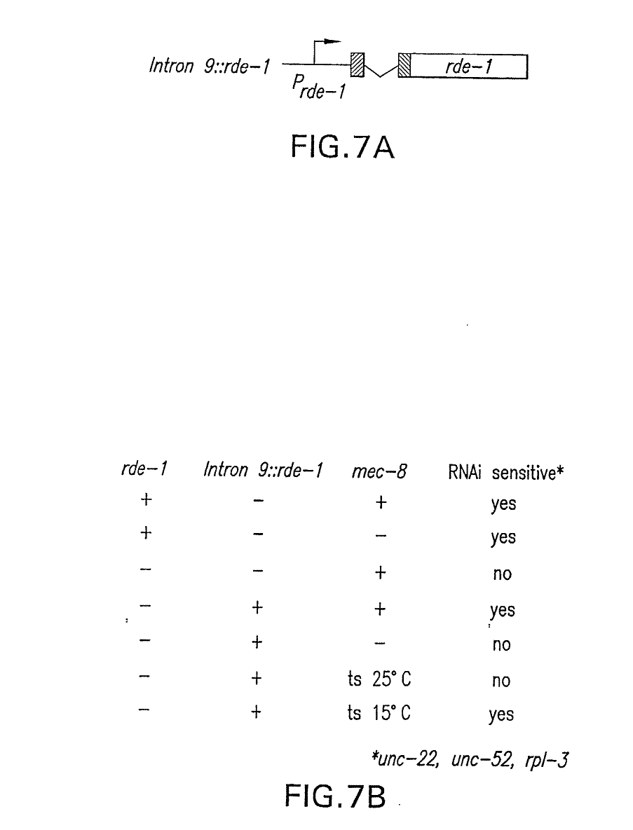

Transformation of mec-8(u218); rde-1 (ne219) (ne219 is a null allele) with P_{rde-1}intron 9::rde-1 (FIG. 7A) resulted in animals that were responsive to RNAi at 15° C. but not at 25° C. (FIG. 7B). Since this temperature-sensitive RNAi phenotype was seen using bacteria making dsRNA for unc-22 (a gene expressed in muscle), unc-52 (a gene expressed in the hypodermis) and rpl-3 (a gene needed for embryonic viability), the RNAi effects may be detected in a variety of tissues and organisms. All the responses observed occured in the P0's, for rde-1 when animals are fed as eggs they show a Gro (growth) defect and never become adults, and when older larvae are fed then they are Ste (sterile) and do not have progeny. For unc-22 the Twitcher phenotype appear one or two days later. Unc-52 is a little more variable, which may be due to the nature of the strain, since in wild type it is also very variable.

Since RDE-1 is thought to act as part of the RISC complex (Tabara et al., 1999; Liu et al., 2004; Hammond et al., 2001), the animals presumably load with dsRNA at the restrictive temperature but cannot execute RNAi. Switching to the permissive temperature may allow RNAi inhibition to proceed, thus making this method particularly useful for the study of late effects of genes whose loss is lethal.

In order to test how quickly the RNAi phenotype could be detected, newly hatched intron 9::rde-1 animals were fed bacteria making dsRNA for unc-22 at 25° C. for 24 hr and then shifted them 15° C. As a further application of this method, a strain that has temperature-dependent RNAi was produced which can be used, for example, to study the function of embryonic lethal genes. To make the strain, mec-8(u218); rde-1 (ne219) animals were transformed with wild-type rde-1 genomic DNA in which the mec-2 INT9 had been inserted just before the first ATG. The resulting transformants are mutant when grown on bacteria making dsRNA for unc-22, unc-52, and rpl-3 at 15° C. but not at 25° C.,

### 7. EXAMPLE 2

The finding that mec-2 INT9 can convey mec-8 dependence suggests that temperature-dependent constructs of any other C. elegans gene can be made. The usefulness of such constructs, however, relies on how faithfully the phenotype of the INT9 construct reflects the generation of the endogenous gene. Certain characteristics of the mec-8 and the mec-8(u218)ts allele support the hypothesis that the intron 9 constructs should mimic this expression. Suppression of an amber allele of mec-8 by tRNA suppressor can be obtained with only a single dose of the suppressor gene (Chalfie and Sulston, 1981), suggesting that a relatively small amount of the wild-type product may be needed for function. This hypothesis is also supported by temperature-shift data for the u218 strain (Chalfie and Au, 1981), specifically that animals shifted from the permissive to restrictive temperature at hatching had sufficient product for adult touch sensitivity. These experiments also suggest that mec-8 displays considerable purdurance.

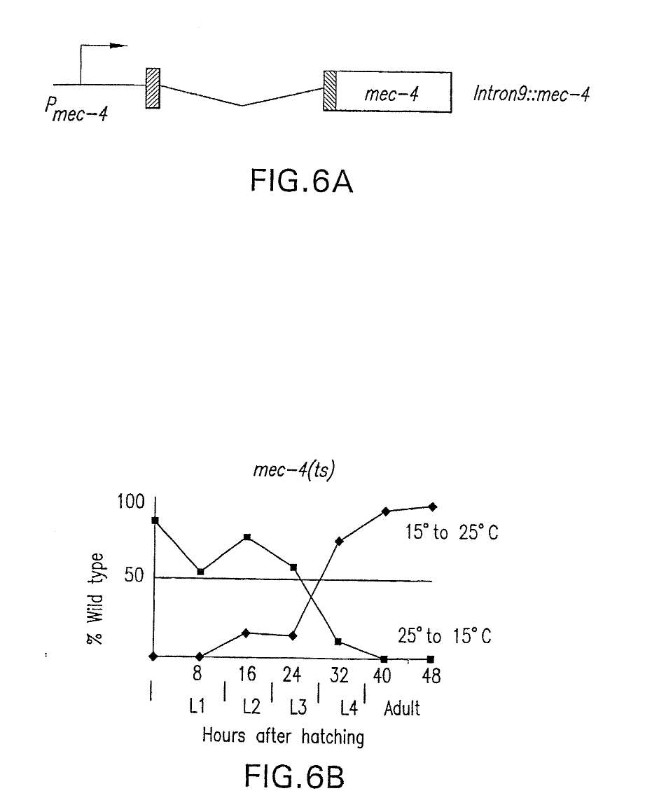

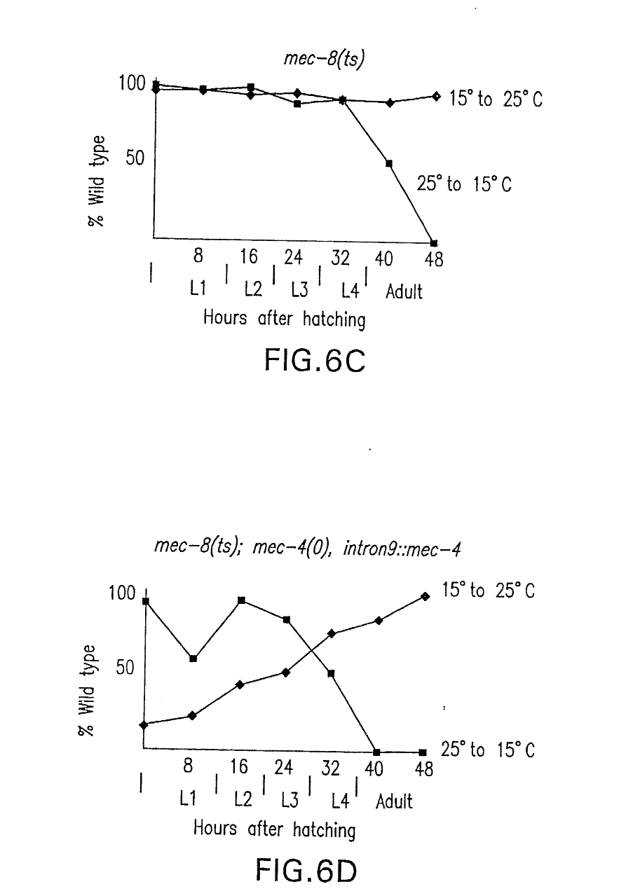

To test whether the use of INT9 and mec-8ts could mimic the results of an endogenous temperature-sensitive mutation, mec-8(u218)ts; mec-4(u253) animals carrying an intron 9 based mec-4 construct driven from the mec-4 promoter (FIG. 6A) were compared with the mec-4(u45)ts animals. The temperature shift curve for mec-8(u218) and nlec-4(u45) are quite different (FIGS. 6C and 6B, respectively)(Chalfie and Au, 1989). However, the temperature shift curve of the intron 9 mec-4 construct is essentially that found for mec-4(u45) (FIG. 6D). This result suggests that the expression of mec-4, but not mec-8, is limiting in these experiments.

### 8. ADDITIONAL REFERENCES

Brenner, S. (1974). The genetics of Caenorhabditis elegans. Genetics 77, 71-94.
Chalfie, M. and Au, M. (1989). Genetic control of differentiation of the Caenorhabditis elegans touch receptor neurons. Science 243, 1027-1033.
Davies, A. G., Spike, C. A., Shaw, J. E. and Herman, R. K. (1999). Functional overlap between the mec-8 gene and five sym genes in Caenorhabditis elegans. Genetics 153, 117-134.
Tabara, H., Sarkissian, M., Kelly, W. G., Fleenor, J., Grishok, A., Timmons, L., Fire, A. and Mello, C. C. (1999) The rde-1 gene, RNA interference, and transposon silencing in C. elegans, Cell 99,123-32.
Okkema, P. G., and Fire, A. (1994). The Caenorhabditis elegans NK-2 class homeoprotein CEH-22 is involved in combinatorial activation of gene expression in pharyngeal muscle. Development 120, 2175-2186.
Mello, C. C., Kramer, J. M., Stinchcomb, D., and Ambros, V. (1991). Efficient gene transfer in C. elegans: extrachromosomal maintenance and integration of transforming sequences. EMBO J. 10, 3959-3970.
Davies, A. G., et al. (1999) Functional overlap between the mec-8 gene and five sym genes in Caenorhabditis elegans. Genetics 153: 117-1134.
Chalfie, M. and J. Sulston (1981) Developmental genetics of the mechanosensory neurons of Caenorhabditis elegans. Dev. Biol. 82: 358-370.
Liu, J., et al. (2004) Argonaute2 is the catalytic engine of mammalian RNAi. Science 305: 1437-1441.
Hammond, S. M., et al. (2001) Argonaute2, a link between genetic and biochemical analyses of RNAi. Science. 293: 1146-1150.

Various publications are cited herein, the contents of which are hereby incorporated by reference in their entireties.

We claim:

1. A nucleic acid comprising an intron cassette/target gene construct comprising a target gene which is not mec-2 interrupted by an INT9 sequence inserted into a region of the target gene upstream of the end of its coding sequence, such that retention of INT9 in a mRNA transcript of the construct would interfere with expression of a functional target gene product.

2. The nucleic acid of claim 1, wherein the intron cassette/target gene construct is operably linked to a promoter sequence.

3. A vector comprising the nucleic acid of claim 1.

4. A vector comprising the nucleic acid of claim 2.

5. A host cell comprising the nucleic acid of claim 1.

6. A host cell comprising the nucleic acid of claim 2.

7. A host cell comprising the nucleic acid of claim 1, further comprising a mec-8 gene which is conditionally expressed as functional MEC-8 protein.

8. The host cell of claim 7, wherein the mec-8 gene is a mutant allele which encodes a MEC-8 protein, the function of which is temperature sensitive.

9. The host cell of claim 7, wherein the mec-8 gene encodes a functional MEC-8 protein, where the transcription of the mec-8 gene is controlled by a conditionally active promoter.

10. The nucleic acid of claim 1, wherein the expression of functional target gene product permits RNAi to interfere with gene expression.

11. The nucleic acid of claim 2, wherein the expression of functional target gene product permits RNAi to interfere with gene expression.

12. The nucleic acid of claim 3, wherein the expression of functional target gene product permits RNAi to interfere with gene expression.

13. The nucleic acid of claim 4, wherein the expression of functional target gene product permits RNAi to interfere with gene expression.

14. The nucleic acid of claim 5, wherein the expression of functional target gene product permits RNAi to interfere with gene expression.

15. The nucleic acid of claim 6, wherein the expression of functional target gene product permits RNAi to interfere with gene expression.

16. The nucleic acid of claim 7, wherein the expression of functional target gene product permits RNAi to interfere with gene expression.

17. The nucleic acid of claim 8, wherein the expression of functional target gene product permits RNAi to interfere with gene expression.

18. The nucleic acid of claim 9, wherein the expression of functional target gene product permits RNAi to interfere with gene expression.

19. A method of controlling expression of a target gene in a cell, comprising:
(i) interrupting a nucleic acid comprising the target gene, which is not mec-2, with an INT9 sequence to form an intron cassette/target gene construct, such that retention of INT9 in a mRNA transcript of the construct would interfere with expression of a functional target gene product;
(ii) operably linking the intron cassette/target gene construct to a promoter;
(iii) expressing the promoter/intron cassette/target gene construct prepared in (ii) in a cell having conditional expression of functional MEC-8; and
(iv) providing conditions which result in expression of functional MEC-8, thereby inducing expression of functional target gene product.

20. The method of claim 19, where the MEC-8 is temperature sensitive.

21. The method of claim 19, where the expression of functional target gene product permits RNAi to interfere with gene expression.

22. A method of rendering expression of a target gene in a cell temperature sensitive, comprising:
(i) interrupting a nucleic acid comprising the target gene, which is not mec-2, with an INT9 sequence to form an intron cassette/target gene construct, such that retention of INT9 in a mRNA transcript of the construct would interfere with expression of a functional target gene product;
(ii) operably linking the intron cassette/target gene construct to a promoter; and
(iii) expressing the promoter/intron cassette/target gene construct prepared in (ii) in a cell having temperature sensitive expression of functional MEC-8;
whereby providing a temperature which results in expression of functional MEC-8 results in expression of a functional target gene product.

23. The method of claim 22, where the expression of functional target gene product permits RNAi to interfere with gene expression.
